(19) United States
(12) Patent Application Publication
(10) Pub. No.: US 2003/0170655 A1
(43) Pub. Date: Sep. 11, 2003

(54) Mus101 and homologue thereof

(76) Inventors: David Moore Glover, Great Gransdon Sandy (GB); Rochele Yamamoto, Providence, RI (US); Daryl Henderson, Stony Brook, NY (US)

Correspondence Address: LAHIVE & COCKFIELD, 28 STATE STREET, BOSTON, MA, 02109, US

(21) Appl. No.: 10/168,424

(22) PCT Filed: Dec. 21, 2000
(86) PCT No.: PCT/GB00/04956

(30) Foreign Application Priority Data
Dec. 24, 1999 (GB) 9930708.4

(51) Int. Cl. C12Q 1/68; G01N 33/53; C07H 21/04; C07K 14/435; C07K 16/18; A61K 38/17; C12P 21/02; C12N 5/06

(52) U.S. Cl. 435/6; 435/7.1; 435/69.1; 435/320.1; 435/325; 530/350; 530/388.1; 536/23.2; 435/348

(57) ABSTRACT

Plynucleotides encoding a novel Drosophila gene product designated mus101 and homologues thereof as well as mus101 polypeptides are provided. Polynucleotide probes derived from the nucleotide sequence of mus101 and antibodies that bind to mus101 protein are also provided as well as assays for identifying substances that regulate mus101 function.

### FIELD OF THE INVENTION

The present invention relates to Drosophila mus101, a member of the BRCT superfamily. The present invention also relates to the use of mus101 and homologues thereof in assays to identify substances capable of disrupting mus101 function

### BACKGROUND TO THE INVENTION

The first screens for mutagen sensitive mutants were performed in the early '70s (Boyd et al., 1976; Boyd et al., 1981; Henderson et al., 1987; Smith, 1976; Snyder and Smith, 1982, for a review, see Boyd et al., 1987). These screens were made with the objective of recovering mutants that displayed increased sensitivity to the monofunctional alkylating agent methyl methanesulphonate (MMS). The mutagenic scheme used in each of these screens was similar, and was based upon mutagenising flies with ethyl methanesulphonate (EMS) and the subsequent recovery of progeny that were sensitive to MMS. Some screens selected mutants that displayed sensitivity to damaging agents other than MMS. Boyd et al., 1981 for example, screened for sensitivity to MMS, N-acetyl-2-aminofluorene (AAF) and nitrogen mustard (HN2) and Henderson et al., 1987 screened for sensitivity to MMS, AAF, HN2 and γ-radiation.

The screens carried out to date have led to the identification of 33 mutagen sensitive loci in the Drosophila genome. The distribution of these loci along the 3 major chromosomes are as follows (Lindsley and Zimm, 1992 or in the flybase web site): 10 on the X-chromosome (mus101, mus102, mus105, mus106, mus108, mus109, mus111, mus112, mei-9 and mei-41), 12 on the second chromosome (mus201-mus211 and phr) and 11 on the third chromosome (mus301, mus302, mus304-mus312). mus101 was isolated in one of the first screens for mutants sensitive to MMS (Boyd et al., 1976). Two EMS-induced mus101 alleles were isolated: mus101D^{D1} and mus101^{D2}. Homozygous and hemizygous larvae from both alleles are sensitive to MMS, HN2, AAF and γ-rays, but are resistant to UV radiation. Other alleles of mus101 showing different phenotypes were later isolated: mus101^{K451} (fs(1)K451) is female sterile (Komitopoulou et at., 1983; Komitopoulou et al., 1988; Orr et al., 1984) and MMS sensitive (Yamamoto, R. R. and Henderson, D. S., unpublished results); mus101^{sm} and mus101^{lcd} are late larval lethals (Axton, 1990); and mus101^{tsl} is a conditional, temperature sensitive lethal allele (Gatti et al., 1983; Smith et al., 1985).

The mutant mus101^{D1} is partially deficient in postreplication repair (Boyd and Setlow, 1976; Brown and Boyd, 1981), as shown by a reduction in the molecular weight of the newly synthesised DNA molecules after UV treatment compared to wild type. Non-irradiated cells from this mutant have a slower rate of gain in molecular weight than control, suggesting a defect in DNA replication (Boyd and Setlow, 1976).

The mutant mus101^{tsl} results in high levels of chromosomal instability, assessed by the presence of mwh clones (Smith et al., 1985). mus101^{tsl} is a hypomorphic allele, since the number of mwh clones is increased in the combination mus101⁵²/Df(1)HA92 (12A6-7, 12D3). The chromosome instability observed genetically is also observed cytologically. Neuroblast cells from homozygous mus101^{tsl} show altered condensation of the heterochromatic region of chromosomes. In this mutant the heterochromatin is abnormally undercondensed, a defect that presents first in the Y chromosome. After 24 hours at the restrictive temperature, heterochromatic regions in the autosomes and X chromosome are also undercondensed and a significant number of cells with broken chromosomes are also seen (Gatti et al., 1983; Smith et al., 1985).

Similar genetic and cytological phenotypes are observed for mus101^{D2}/Df(1)HA92 and mus101^{D2}/mus101^{tsl} (Smith et al., 1985). Chromosomal instability has also been observed genetically for homozygous mus101^{D2} females (Baker and Smith, 1979).

The fact that no cytological defect has been observed in ganglia from homozygous mus101^{D2} females (Gatti, 1979) suggests that this is a hypomorphic allele.

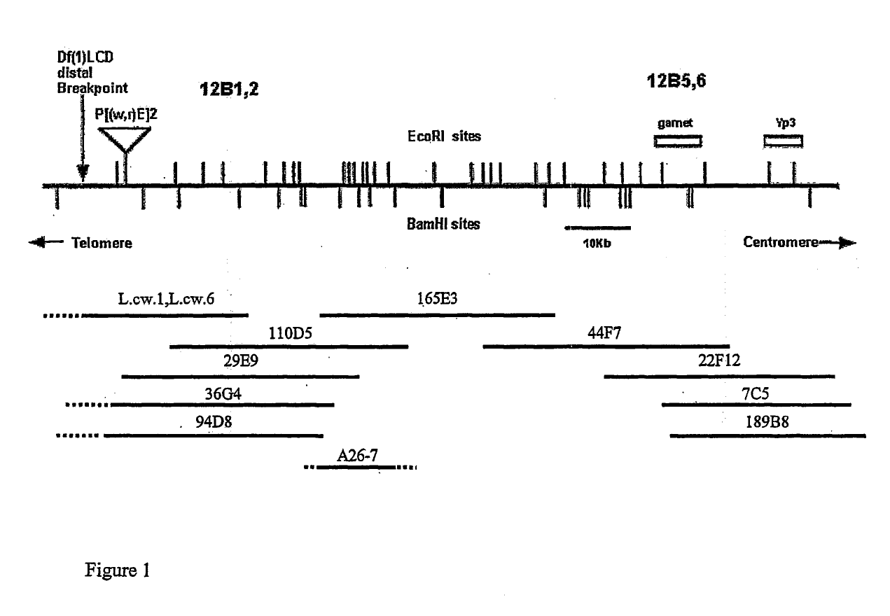

Analysis of neuroblast cells from mus101^{lcd} also show irregularities in the heterochromatic region, represented by undercondensation of pericentric heterochromatin (FIG. 1.1B and C). Chromosome fragmentation is also observed (Axton, 1990).

The female sterile allele mus101^{k451} was isolated in a screen for mutants affecting eggshell formation (Komitopoulou et al., 1983). Homozygous females for this mutation have reduced viability and produce flaccid eggs, with thin eggshells and small dorsal appendages (Komitopoulou et al., 1983; Komitopoulou et al., 1988).

The chorion defects observed in mus101^{k451} are due to a reduced level of the major chorion proteins (Komitopoulou et al., 1988). This in turn is due to a reduced amplification of the chorion genes, specially those located on the third chromosome (Orr et al., 1984). The amplification of chorion genes is a specific form of DNA replication that occurs in follicle cells and is essential for rapid eggshell formation. Two major gene clusters undergo amplification: one on the X chromosome, and one on the third chromosome (for review see Orr-Weaver, 1991). mus101⁺ regulates chorion gene amplification in trans (Orr et al., 1984).

The phenotypes of the various mus101 alleles sumllmarised above suggest a role for the, mus101⁺ gene product in different aspects of chromosome maintenance. Mus101 protein can be involved in the postreplication repair of DNA (mus101^{D1}), DNA replication (mus101^{K451}), chromatin condensation and chromosome stability (mus101^{tsl}, mus101^{D2} and mus101^{lcd}). Gatti et al., 1983 suggested that the mutagen-sensitivity and repair-defective phenotypes shown by viable mus101 alleles are secondary consequences of a primary effect on chromosome condensation that either renders chromatin more susceptible to mutagen damage or less available to repair. The opposite is also possible. Defects in DNA replication could lead to the heterochromatin undercondensation observed in some mutants. It is also possible that Mus101 protein is involved in different cellular processes, if it associates with different proteins to perform different tasks. It is evident that the molecular cloning of such an interesting: gene should bring more insight into the function of its encoded protein.

### SUMMARY OF THE INVENTION

We have now identified isolated genomic and cDNA clones encoding mus101, enabling the deduction of the Mus101 amino acid sequence and recombinant expression of Mus101 protein.

Accordingly, the invention provides a mus101 polypeptide or a homologue thereof. The polypeptide preferably has one or more of the additional features:

(1) one or more BRCT domains, preferably five or more BRCT domains, more preferably seven BRCT domains;

(2) a molecular mass of from x to y kDa, as determined by SDS-PAGE;

(3) binds to topoisomerase IT or a homologue thereof, more preferably Topo II β,

(4) localises to the spindle poles.

Preferably the polypeptide is encoded by a cDNA sequence obtainable from a eukaryotic cDNA library, preferably a metazoan cDNA library (such as insect or mammalian) said DNA sequence comprising a DNA sequence being selectively detectable with a Drosophila mus101 nucleotide sequence as shown in SEQ ID No. 1 or a fragment thereof.

The term “selectively detectable” means that the cDNA used as a probe is used under conditions where a target cDNA of the invention is found to hybridize to the probe at a level significantly above background. The background hybridization may occur because of other cDNAs present in the cDNA library. In this event background implies a level of signal generated by interaction between the probe and a non-specific cDNA member of the library which is less than 10 fold, preferably less than 100 fold as intense as the specific interaction observed with the target cDNA. The intensity of interaction may be measured, for example, by radiolabelling the probe, e.g. with ³²P. Suitable conditions may be found by reference to the Examples.

The invention also provides the mus101 polypeptide of SEQ ID. 2 and derivatives, variants homologues thereof, polypeptide fragments thereof, as well as antibodies capable of binding the mus101 protein or polypeptide fragments thereof.

In another aspect, the present invention provides a polynucleotide selected from:

(a) polynucleotides comprising the nucleotide sequence set out in SEQ ID No. 1 or the complement thereof.

(b) polynucleotides comprising a nucleotide sequence capable of hybridising to the nucleotide sequence set out in SEQ ID No. 1, or a fragment thereof.

(c) polynucleotides comprising a nucleotide sequence capable of hybridising to the complement of the nucleotide sequence set out in SEQ ID No. 1 or a fragment thereof.

(d) polynucleotides comprising a polynucleotide sequence which is degenerate as a result of the genetic code to the polynucleotides defined in (a), (b) or (c).

Also provided are polynucleotides encoding polypeptides of the invention. All such polynucleotides will be referred to as a polynucleotide of the invention. A polynucleotide of the invention includes a polynucleotide having a sequence as shown in SEQ ID No. 1 and fragments thereof capable of selectively hybridising to the mus101 gene.

In a further aspect, the invention provides recombinant vectors carrying a polynucleotide of the invention, including expression vectors, and methods of growing such vectors in a suitable host cell, for example under conditions in which expression of a protein or polypeptide encoded by a sequence of the invention occurs.

In an additional aspect, the invention provides kits comprising polynucleotides, polypeptides or antibodies of the invention and methods of using such kits in diagnosing the presence of absence of mus101 and its homologues, or variants thereof, including deleterious mutants.

In a further aspect, the present invention provides the use of a mus101 polypeptide or homologue, derivative, variant or fragment thereof in a method of identifying a substance capable of affecting mus101 function. For example, the invention provides the use of a mus101 polypeptide or homologue, derivative, variant or fragment thereof in an assay for identifying a substance capable of increasing the susceptibility of a cell to DNA damaging agents. Other possible mus101 functions for which it may be desired to identify substances which affect such functions include DNA repair and cell cycle regulation.

In this respect, the invention also provides a method for identifying a substance capable of binding to a mus101 polypeptide or a homologue, derivative, variant or fragment thereof, which method comprises incubating the mus101 polypeptide or homologue, derivative, variant or fragment thereof with a candidate substance and determining whether the substance binds to the mus101 polypeptide or homologue, derivative, variant or fragment thereof

Also provided is a substance identified by the above methods of the invention. Such substances may be used in a method of therapy, such as in a method of affecting mus101 function, in particular, increasing the susceptibility of a cell to DNA damaging agents.

The invention also provides a process comprising the steps of:

(a) performing one of the above methods; and

(b) preparing a quantity of those one or more substances identified as being capable of binding to a mus101 polypeptide or homologue, derivative, variant or fragment thereof.

Also provided is a process comprising the steps of:

(a) performing one of the above method; and

(b) preparing a pharmaceutical composition comprising one or more substances identified as being capable of binding to a mus101 polypeptide or homologue, derivative, variant or fragment thereof.

The present invention further provides a method of treating a tumour comprising administering to a patient in need of treatment an effective amount of a polynucleotide, polypeptide or antibody of the invention.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al., Current Protocols in Molecular Biology (1999) 4^{th} Ed, John Wiley & Sons, Inc.

A. Polypeptides

It will be understood that polypeptides of the invention are not limited to polypeptides having the amino acid sequence set out in SEQ. ID. No. 2 or fragments thereof but also include homologous sequences obtained from any source, for example related viral/bacterial proteins, cellular homologues and synthetic peptides, as well as variants or derivatives thereof.

Thus polypeptides of the invention also include those encoding mus101 homologues from other species including animals such as mammals (e.g. mice, rats or rabbits), especially primates, more especially humans. More specifically, Mus101 homologues included within the scope of the invention for use in assays and methods of treatment include human mus101.

Thus, the present invention covers variants, homologues or derivatives of the amino acid sequence set out in SEQ ID No. 2 of the present invention, as well as variants, homologues or derivatives of the nucleotide sequence coding for the amino acid sequences of the present invention.

In the context of the present invention, a homologous sequence is taken to include an amino acid sequence which is at least 15, 20, 25, 30, 40, 50, 60, 70, 80 or 90% identical, preferably at least 95 or 98% identical at the amino acid level over at least 50 or 100, preferably 200, 300, 400 or 500 amino acids with SEQ ID No. 2. In particular, homology should typically be considered with respect to those regions of the sequence known to be essential for protein function rather than non-essential neighbouring sequences. This is especially important when considering homologous sequences from distantly related organisms. Details of particular comparisons between Drosophila mus101 and human TopBP1 are given in the Examples and indicate that amino acid homology (identity) can be as low as about 25 or 30% over the complete sequence or a fragment comprising BRCT domains. Homology may also be considered with respect to the homologous human sequences described below.

Particularly preferred regions over which to conduct homology comparisons are the BRCT domains amino acids X and/or Y of SEQ ID. No. 2. Another important region is the region showing homology to human treacle protein (TCOF1) region (amino acids 820 to 956 of SEQ I.D. No.2). Preferably homology to this region is at least 25%, more preferably at least 30 or 35%.

Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate %.homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap, scoring system. High gap penalties will of course produce optimised alignments with fewer gaps. Most alignment programs allow the gap penalties to be modified. However, it is preferred to use the default values when using such software for sequence comparisons. For example when using the GCG Wisconsin. Bestfit package (see below) the default gap penalty for amino acid sequences is −12 for a gap and −4 for each extension

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. .Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result

The terms “variant” or “derivative” in relation to the amino acid sequences of the present invention includes any substitution of, variation of, modification of, replacement of, deletion of or addition of one :(or more) amino acids from or to the sequence providing the resultant amino acid sequence retains substantially the same activity as the unmodified sequence, preferably having at least the same activity as the polypeptides presented in the sequence listings.

Polypeptides having the amino acid sequence shown in SEQ I.D. No. 2, or fragments or homologues thereof may be modified for use in the present invention. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides of the invention. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Polypeptides of the invention also include fragments of the fall length sequences mentioned above. Preferably said fragments comprise at least one epitope. Fragments will typically comprise at least 6 amino acids, more preferably at least 10, 20, 30, 50 or 100 amino acids. Preferred fragments comprise functional domains of the full length mus101 polypeptide (such as the BRCT domains described above), for example fragments capable of binding to topoisomerase II. In particular, preferred fragments comprise the seven BRCT domains.

In a particularly preferred aspect of the invention, the full length human TopBP1 protein sequence described by Yamane et al., 1997, is specifically excluded from the scope of the term “polypeptides of the invention”. The fall length sequences of this protein is available under Accession no. AB019397—gi 3845612. However, it is preferred that mus101 polypeptide homologues within the scope of the invention include polypeptides having less than 99, 98, 95 or 90% homology but more than 30, 40 or 50% homology to the full length sequences set out as Accession no. AB019397.

Proteins of the invention are typically made by recombinant means, for example as described below. However they may also be made by synthetic means using techniques well known to skilled persons such as solid phase synthesis. Proteins of the invention may also be produced as fusion proteins, for example to aid in extraction and purification. Examples of fusion protein partners include glutathione-S-transferase (GST), 6×His, GAM4 (DNA binding and/or transcriptional activation domains) and P-galactosidase. It may also be convenient to include a proteolytic cleavage site between the fusion protein partner and the protein sequence of interest to allow removal of fusion protein sequences. Preferably the fusion protein will not hinder the function of the protein of interest sequence.

Proteins of the invention may be in a substantially isolated form. It will be understood that the protein may be mixed with carriers or diluents which will not interfere with the intended purpose of the protein and still be regarded as substantially isolated. A protein of the invention may also be in a substantially purified form, in which case it will generally comprise the protein in a preparation in which more than 90%, e.g. 95%, 98% or 99% of the protein in the preparation is a protein of the invention.

A polypeptide of the invention may be labelled with a revealing label. The revealing label may be any suitable label which allows the polypeptide to be detected. Suitable labels include radioisotopes, e.g. ¹²⁵I, enzymes, antibodies, polynucleotides and linkers such as biotin. Labelled polypeptides of the invention may be used in diagnostic procedures such as immunoassays to determine the amount of a polypeptide of the invention in a sample. Polypeptides or labelled polypeptides of the invention may also be used in serological or cell-mediated immune assays for the detection of immune reactivity to said polypeptides in animals and humans using standard protocols.

A polypeptide or labelled polypeptide of the invention or fragment thereof may also be fixed to a solid phase, for example the surface of an immunoassay well or dipstick. Such labelled and/or immobilised polypeptides may be packaged into kits in a suitable container along with suitable reagents, controls, instructions and the like. Such polypeptides and kits may be used in methods of detection of antibodies to the mus101 polypeptides or their allelic or species variants by immunoassay.

Immunoassay methods are well known in the art and will generally comprise:

(a) providing a polypeptide comprising an epitope bindable by an antibody against said protein;

(b) incubating a biological sample with said polypeptide under conditions which allow for the formation of an antibody-antigen complex; and

(c) determining whether antibody-antigen complex comprising said polypeptide is formed.

Polypeptides of the invention may be used in in vitro or in vivo cell culture systems to study the role of mus101 and its homologues in disease. For example, truncated or modified mus101 may be introduced into a cell to disrupt the normal functions which occur in the cell. Specific examples may include fragments of mus101 or its homologues which comprise only the BRCT domains, such as BRCT I, II, II, IV, V, VI or VII or any combination thereof. The polypeptides of the invention may be introduced into the cell by in situ expression of the polypeptide from a recombinant expression vector (see below). The expression vector optionally carries an inducible promoter to control the expression of the polypeptide.

The use of higher eukaryotic, such as insect or mammalian, host cells is expected to provide for such post-translational modifications (e.g. myristolation, glycosylation, truncation, lapidation and tyrosine, serine or threonine phosphorylation) as may be needed to confer optimal biological activity on recombinant expression products of the invention. Such cell culture systems in which polypeptides of the invention are expressed may be used in assay systems to identify candidate substances which interfere with or enhance the functions of the polypeptides of the invention in the cell.

B. Polynucleotides

Polynucleotides of the invention include polynucleotides comprising the nucleic acid sequence set out in SEQ ID No. 1 and fragments thereof Polynucleotides of the invention also include polynucleotides encoding the polypeptides of the invention. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

Polynucleotides of the invention may comprise DNA or RNA. They may be single-stranded or double-stranded They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

The terms “variant”, “homologue” or “derivative” in relation to the nucleotide sequence of the present invention include any substitution of; variation of, modification of, replacement of, deletion of or addition of one (or more) nucleic acid from or to the sequence. Preferably said variant, homologues or derivatives code for a polypeptide having biological activity, preferably having substantially the same activity as the amino acid sequence shown as SEQ ID No. 2.

In a particularly preferred aspect of the invention, the full length human TopBP1 nucleotide sequence described by Yamane et at., 1997, is specifically excluded from the scope of the term “polynucleotides of the invention”. The full length polynucleotide sequence is available under Accession no. AB019397—gi 3845612. However, it is preferred that homologues within the scope of the invention include nucleotides having less than 99, 98, 95 or 90% homology but more than 30, 40 or 50% homology to the full length sequences set out in Accession no. AB019397.

As indicated above, with respect to sequence homology, preferably there is at least 50 or 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and -9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides of the invention capable of selectively hybridising to the nucleotide sequences presented herein, or to their .complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred polynucleotides of the invention will comprise regions encoding polypeptide domains homologous to the polypeptide domains described above (for example the BRCT domains), preferably at least 70, 80 or 90% and more preferably at least 95% homologous to said regions.

The term “selectively hybridizable” means that the polynucleotide used as a probe is used under conditions where a target polynucleotide of the invention is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC-{1×SSC=0.15 M NaCl, 0.015 MNa₃Citrate pH 7.0).

Where the polynucleotide of the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of SEQ I.D. No 1 under conditions of medium to high stringency. More preferably, the nucleotide sequences of the human TopBP1 protein described by Yamane et al., 1997 (Accession no. AB01939i), or fragments thereof, may be used to identify other primate/mammalian homologues since nucleotide homology between human sequences and mammalian sequences is likely to be higher than is the case for the Drosophila sequence identified herein.

Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by-aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments of SEQ I.D. No. 1.

Alternatively, such polynucleotides may be obtained by site directed mutagenesis of characterised sequences, such as SEQ ID. No 1. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides. For example, further changes may be desirable to represent particular coding changes found in mus101 which give rise to mutant mus101 genes which have lost their regulatory function. Probes based on such changes can be used as diagnostic probes to detect such mus101 mutants.

Polynucleotides of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as a DNA polynucleotides and probes according to the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites, so that the amplified DNA can be cloned into a suitable cloning vector

Polynucleotides or primers of the invention may carry a revealing label. Suitable labels include radioisotopes such as ³²P or 35S, enzyme labels, or other protein labels such as biotin. Such labels may be added to polynucleotides or primers of the invention and may be detected using by techniques known per se.

Polynucleotides or primers of the invention or fragments thereof labelled or unlabelled may be used by a person skilled in the art in nucleic acid-based tests for detecting or sequencing mus101 and its homologues in the human or animal body.

Such tests for detecting generally comprise bringing a biological sample containing DNA or RNA into contact with a probe comprising a polynucleotide or primer of the invention under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO89/03891 and WO90/13667.

Tests for sequencing mus101 and its homologues include bringing a biological sample containing target DNA or RNA into contact with a probe comprising a polynucleotide or primer of the invention under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; tall, owing strand elongation and termination reaction to occur, separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Tests for detecting or sequencing mus101, or its homologue, in a biological sample may be used to determine mus101 sequences within cells in individuals who have, or are suspected to have, an altered mus101 gene sequence, for example within cancer cells including leukaemia cells and solid tumours such as breast, ovary, lung, colon, pancreas, testes, liver, brain, muscle and bone tumours.

In addition, the discovery of mus101 will allow the role of this gene in hereditary diseases to be investigated. In general, this will involve establishing the status of mus101, or its homologue (e.g. using PCR sequence analysis), in cells derived from animals or humans with, for example, tumours.

The probes of the invention may conveniently be packaged in the form of a test kit in a suitable container. In such kits the probe may be bound to a solid support where the assay format for which the kit is designed requires such binding. The kit may also contain suitable reagents for treating the sample to be probed, hybridising the probe to nucleic acid in the sample, control reagents, instructions, and the like.

C. Nucleic Acid Vectors

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of a protein of the invention. This process may comprise culturing a host cell transformed with an expression vector as described above under conditions to provide for expression by the vector of a coding sequence encoding the protein, and optionally recovering the expressed protein. Vectors will be chosen that are compatible with the host cell used.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the polypeptide of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells, such as insect cells, may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Polynucleotides according to the invention may also be inserted into the vectors described above in an antisense orientation to provide for the production of antisense RNA. Antisense RNA or other antisense polynucleotides may also be produced by synthetic means. Such antisense polynucleotides may be used in a method of controlling the levels of mus101 or its variants or species homologues.

D. Host Cells

Vectors and polynucleotides of the invention may be introduced into host cells for the purpose of replicating the vectors/polynucleotides and/or expressing the polypeptides of the invention encoded by the polynucleotides of the invention. Although the polypeptides of the invention may be produced using prokaryotic cells as host cells, it is preferred to use eukaryotic cells, for example yeast, insect or mammalian cells, in particular mammalian cells.

Vectors/polynucleotides of the invention may be introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

E. Protein Expression and Purification

Host cells comprising polynucleotides of the invention may be used to express polypeptides of the invention. Host cells may be cultured under suitable conditions which allow expression of the proteins of the invention. Expression of the polypeptides of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Polypeptides of the invention can be extracted from host cells by a variety of techniques known in the art, including enzymatic, chemical and/or osmotic lysis and physical disruption.

Polypeptides of the invention may also be produced recombinantly in an in vitro cell-free system, such as the TnT™ (Promega) rabbit reticulocyte system.

F. Antibodies.

The invention also provides monoclonal or polyclonal antibodies to polypeptides of the invention or fragments thereof. Thus, the present invention further provides a process for the production of monoclonal or polyclonal antibodies to polypeptides of the invention.

If polyclonal antibodies are desired, a selected mammal (e.g., mouse, rabbit, goat, horse, etc.) is immunised with an immunogenic polypeptide bearing a mus101 epitope(s). Serum from the immunised animal is collected and treated according to known procedures. If serum containing polyclonal antibodies to an mus101 epitope contains antibodies to other antigens, the polyclonal antibodies can be purified by immunoaffinity chromatography. Techniques for producing and processing polyclonal antisera are known in the art. In order tat such antibodies may be made, the invention also provides polypeptides of the invention or fragments thereof haptenised to another polypeptide for use as immunogens in animals or humans.

Monoclonal antibodies directed against mus101 epitopes in the polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against mus101 epitopes can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against mus101 epitopes are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the pinposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies.(scFv). Furthermore, the antibodies and fragments thereof may be humanised antibodies, for example as described in EP-A-239400.

Antibodies may be used in method of detecting polypeptides of the invention present in biological samples by a method which comprises:

(a) providing an antibody of the invention;

(b) incubating a biological sample with said antibody under conditions which allow for the formation of an antibody-antigen complex; and

(c) determining whether antibody-antigen complex comprising said antibody is formed.

Suitable samples include extracts tissues such as brain, breast, ovary, lung, colon, pancreas, testes, liver, muscle and bone tissues or from neoplastic growths derived from such tissues.

Antibodies of the invention may be bound to a solid support and/or packaged into kits in a suitable container along with suitable reagents, controls, instructions and the like.

G. Assays

The present invention provides assay that are suitable for identifying substances that bind to mus101 polypeptides (reference to which includes homologues, variants, derivatives and fragments as described above). In addition, assays are provided that are suitable for identifying substances that interfere with mus101 binding to components of the cellular machinery such as topoisomerase II. Such assays are typically in vitro. Assays are also provided that test the effects of candidate substances identified in preliminary in vitro assays on intact cells in whole cell assays.

Candidate Substances

A substance that inhibits cell division (including mitosis and/or meiosis) as a result of an interaction with mus101 polypeptides may do so in several ways. It may directly disrupt the binding of mus101 to a component of the spindle apparatus by, for example, binding to mus101 and masking or altering the site of interaction with the other component. Candidate substances of this type may conveniently be preliminarily screened by in vitro binding assays as, for example, described below and then tested, for example in a whole cell assay as described below. Examples of candidate substances include antibodies which recognise mus101 and peptides derived from topoisomerase II polypeptides.

A substance which can bind directly to mus101 may also inhibit its function in cell division by altering its subcellular localisation thus preventing mus101 and components of the mitotic apparatus from coming into contact within the cell. This can be tested using, for example the whole cells assays described below. Non-functional homologues of mus101 may also be tested for inhibition of mitosis since they may compete with mus101 for binding to components of the mitotic apparatus whilst being incapable of the normal functions of mus101 or block the function of mus101 bound to the mitotic apparatus. Such non-functional homologues may include naturally occurring mus101 mutants and modified mus101 sequences or fragments thereof. In particular, fragments of mus101 which comprise one or more BRCT domains but lack other functional domains may be used to compete with full length mus101 for binding to cellular components.

Alternatively, instead of preventing the association of the components directly, the substance may suppress the biologically available amount of mus101. This may be by inhibiting expression of the component, for example at the level of transcription, transcript stability, translation or post-translational stability. An example of such a substance would be antisense RNA or double-stranded interfering RNA sequences which suppresses the amount of mus101 mRNA biosynthesis.

Suitable candidate substances include peptides, especially of from about 5 to 30 or 10 to 25 amino acids in size, based on the sequence of the various domains of Drosophila mus101 described in section A, or variants of such peptides in which one or more residues have been substituted. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for mus101. Furthermore, combinatorial libraries, peptide and peptide mimetics, defined chemical entities, oligonucleotides, and natural product libraries may be screened for activity as inhibitors of binding of mus101 to cellular components such as topoisomerase II. The candidate substances may be used in an initial screen in batches of, for example 10 substances per reaction, and the substances of those batches which show inhibition tested individually. Candidate substances which show activity in in vitro screens such as those described below can then be tested in whole cell systems, such as mammalian cells which will be exposed to the inhibitor and tested for inhibition of mitosis or increased sensitivity to DNA damaging agents.

Mus101 Binding Assays

One type of assay for identifying substances that bind to Mus101 involves contacting an mus101 polypeptide, which is immobilised on a solid support, with a non-immobilised candidate substance determining whether and/or to what extent the, mus101 polypeptide and candidate substance bind to each other. Alternatively, the candidate substance may be immobilised and the mus101 polypeptide non-immobilised.

In a preferred assay method, the mus101 polypeptide is immobilised on beads such as agarose beads. Typically this is achieved by expressing the component as a GST-fusion protein in bacteria, yeast or higher eukaryotic cell lines and purifying the GST-fusion protein from crude cell extracts using glutathione-agarose beads (Smith and Johnson, 1988). As a control, binding of the candidate substance, which is not a GST-fusion protein, to the immobilised mus101 polypeptide is determined in the absence of the mus101 polypeptide. The binding of the candidate substance to the immobilised mus101 polypeptide is then determined. This type of assay is known in the art as a GST pulldown assay. Again, the candidate substance may be immobilised and the mus101 polypeptide non-immobilised.

It is also possible to perform this type of assay using different affinity purification systems for immobilising one of the components, for example Ni-NTA agarose and histidine-tagged components.

Binding of the mus101 polypeptide to the candidate substance may be determined by a variety of methods well-known in the art. For example, the non-immobilised component may be labelled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

Interacting proteins including components of multimeric protein complexes involving mus101 may also be identified by, for example, a two-hybrid screen. The two-hybrid system was developed in yeast (Chien et al., 1991, Proc. Natl. Acad Sci USA 88, 9578-9582) and is based on functional in vivo reconstitution of a transcription factor which activates a reporter gene Specifically, a polynucleotide encoding a protein that interacts with mus101 is isolated by: transforming or transfecting appropriate host cells with a DNA construct comprising a reporter gene under the control of a promoter regulated by a transcription factor having DNA a binding domain and an activating domain; expressing in the host cells a first hybrid DNA sequence encoding a first fusion of part or all of mus101 and either the DNA binding domain or the activating domain of the transcription factor; expressing in the host cell a library of second hybrid DNA sequences encoding second fusion of part or all putative mus101 binding proteins and the DNA binding domain or activating domain of the transcription factor which is not incorporated in the first fusion; detecting binding of an mus101 interacting protein to mus101 in a particular host cell by detecting the production of reporter gene product in the host cell; and isolating second hybrid DNA sequences encoding the interacting protein from the particular host cell. Presently preferred for use in the assay a lexA promoter to drive expression of the reporter gene, the lacZ reporter gene, a transcription factor comprising the lexA DNA binding domain and the GAL4 transactivation domain, and yeast host cells.

Assays for identifying compounds that modulate interaction of mus101 with other proteins may involve: transforming or transfecting appropriate host cells with a DNA construct comprising a reporter gene under the control of a promoter regulated by a transcription factor having a DNA-binding domain and an activating domain; expressing in the host cells a first hybrid DNA sequence encoding a first fusion of part or all of mus101 and the DNA binding domain or the activating domain of the transcription factor; expressing in the host cells a second hybrid DNA sequence encoding part or all of a protein that interacts with mus101 and the DNA binding domain or activating domain of the transcription factor which is not incorporated in the first fusion; evaluating the effect of a test compound on the interaction between mus101 and the interacting protein by detecting binding of the interacting protein to mus101 in a particular host cell by measuring the production of reporter gene product in the host cell in the presence or absence of the test compound; and identifying modulating compounds as those test compounds altering production of the reported gene product in comparison to production of the reporter gene product in the absence of the modulating compound. Presently preferred for use in the assay are a lexA promoter to drive expression of the reporter gene, the lacZ reporter gene, a transcription factor comprising the lexA DNA domain and the GAL4 transactivation domain, and yeast host cells.

Another type of assay for identifying compounds that modulate the interaction between mus101 and an interacting protein involves immobilising mus101 or a natural mus101 interacting protein, detectably labelling the nonimmobilised binding partner, incubating the binding partners together and determining the effect of a test compound on the amount of label bound wherein a reduction in the label bound in the present of the test compound compared to the amount of label bound in the absence of the test compound indicates that the test agent is an inhibitor of mus101 interaction with the protein. Conversely, an increase in the bound in the presence of the test compared to the amount label bound in the absence of the compared indicates that the putative modulator is an activator of mus101 interaction with the protein.

Whole Cell Assays

Candidate substances may also be tested on whole cells for their effect on cell division, including mitosis and/or meiosis. Alternatively, or in addition, candidate substances may be tested for their ability to increase the susceptibility of a cell to DNA damaging agents. Preferably the candidate substances have been identified by the above-described in vitro methods. Alternatively, rapid throughput screens for substances capable of inhibiting cell division, typically mitosis, and/or increasing susceptibility to DNA damaging agents may be used as a preliminary screen and then used in the in vitro assay described above to confirm that the affect is on mus101.

The candidate substance, i.e. the test compound, may be administered to the cell in several ways. For example, it may be added directly to the cell culture medium or injected into the cell. Alternatively, in the case of polypeptide candidate substances, the cell may be transfected with a nucleic acid construct which directs expression of the polypeptide in the cell. Preferably, the expression of the polypeptide is under the control of a regulatable promoter.

Typically, an assay to determine the effect of a candidate substance, such as a candidate substance identified by the method of the invention, on cell mitosis comprises administering the candidate substance to a cell and determining whether the substance inhibits mitosis. Techniques for measuring mitosis in a cell population are well known in the art. The extent of mitosis in treated cells is compared with the extent of mitosis in an untreated control cell population to determine the degree of inhibition, if any.

Typically an assay to determine the effect of a candidate substance, such as a candidate substance identified by the method of the invention, on the susceptibility of cells to DNA damaging agents, comprises administering the candidate substance to a cell, treating the cell with a DNA damaging agent and determining whether the substance increases the susceptibility of the cell to DNA damage. Techniques for treating cells and measuring susceptibility to DNA damage (such as treating cells with X-rays and measuring cell survival) are well known in the art. Typically, the extent of cell death in a treated cell population is compared with the extent of cell death in an untreated control cell population to determine the increase in susceptibility if any. A suitable protocol is described in Errami et al., 1998, Nucl. Acids Res. 26: 4332-4338. DNA damaging agents include MMS, mitomycin C, alkylating agents such as methyl methanesulphonate and ethyl methanesulphonate, bleomycin, X-rays (typically use up to 10 Gy), gamma rays, UV light (typically use up to 20 J/m²)

The concentration of candidate substances used will typically be such that the final concentration in the cells is similar to that described above for the in vitro assays.

A candidate substance is typically considered to be an inhibitor of mitosis if mitosis is reduced to below 50%, preferably below 40, 30, 20 or 10% of that observed in untreated control cell populations. Increased sensitivity to DNA damaging agents typically results in a 10-fold, preferably a 100-fold reduction in survival of treated cells relative to untreated cells.

H. Therapeutic Uses

Many tumours are associated with rapid and often aberrant mitosis. One therapeutic approach to treating cancer is to inhibit mitosis in rapidly dividing cells. Thus, since mus101 appears to be required for the normal mitotic process, it represents a target for inhibition of cell division, particularly in tumour cells. In addition, since mus101 appears to be involved in post-replicative repair and mus101 mutants show increased susceptibility to DNA damaging agents, disruption of mus101 function may be used in conjunction with chemotherapy and/or radiotherapy to increase the susceptibility of tumour cells to the chemotherapy and/or radiotherapy.

One possible approach is to express anti-sense mus101 constructs, preferably selectively in tumour cells, to inhibit mus101 function. Another approach is to use non-functional variants of mus101 that compete with mus101 for cellular components of mitosis, resulting in inhibition of mitosis. Alternatively, compounds identified by the assays described above as binding to mus101 may be administered to tumour cells to prevent mus101 function. This may be performed, for example, by means of gene therapy or by direct administration of the compounds. Anti-mus101 antibodies may also be used as therapeutic agents.

I. Administration

Substances identified or identifiable by the assay methods of the invention may preferably be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding polypeptide components (or antisense constructs) for use in inhibiting mitosis may be administered directly as a naked nucleic acid construct. They may further comprise flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg. It is particularly preferred to use polynucleotides/vectors that target specifically tumour cells, for example by virtue of suitable regulatory constructs or by the use of targeted viral vectors.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide or vector according to the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The routes of administration and dosages described are intended only as a guide since a skilled practitioner will be able to determine readily the optimum route of administration and dosage for any particular patient and condition.

The invention will now be further described by way of Examples, which are meant to serve to assist one of ordinary skill in the art in carrying out the invention and are not intended in any way to limit the scope of the invention The Examples refer to the Figures. In the Figures:

### DETAILED DESCRIPTION OF THE FIGURES

FIG. 1—Molecular map of the 12B region.

The region 12B is represented by 150 kb of cloned genomic DNA. The cosmids and phages used to cover the walk in the region 12B are represented in the lower part of the map. Restriction sites for endonucleases EcoRI and BamHI are represented in the upper and lower parts of the map. Important landmarks in the walk are represented. See text for details.

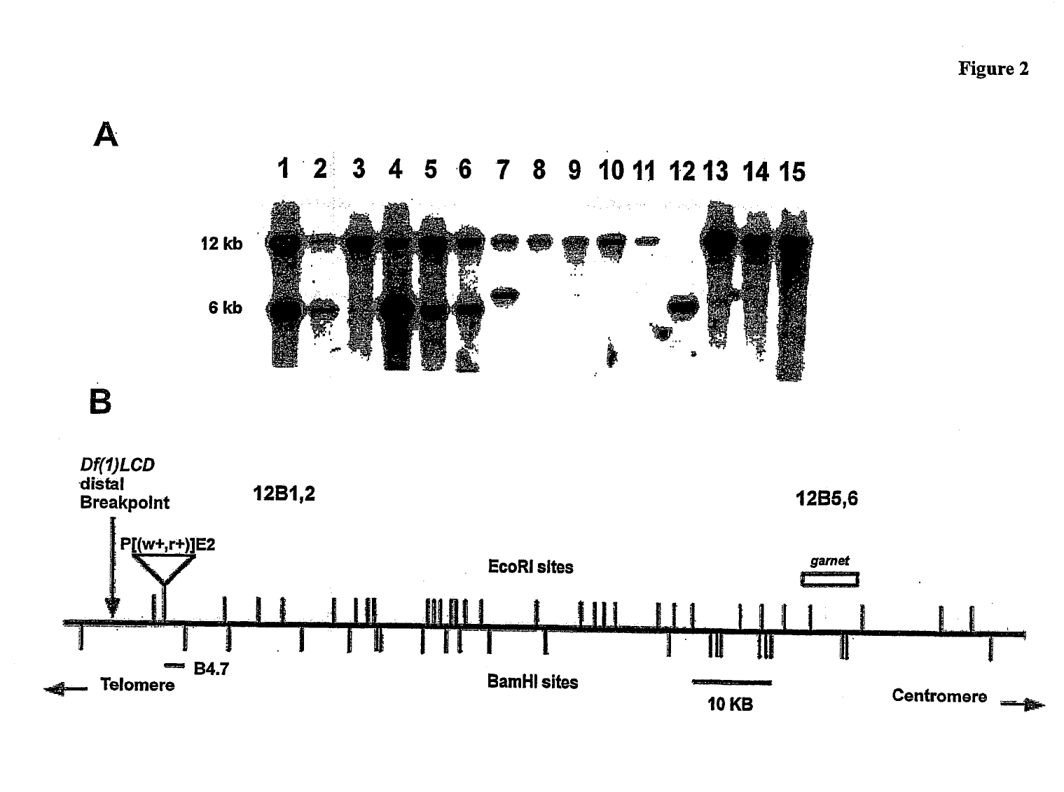

FIG. 2—Molecular analysis of X-ray induced, DEB-induced and P-element imprecise excision mutants recovered in this work.

The deletion of the chromosome walk was monitored by the absence of a 6 kb BamHI fragment revealed after hybridization with probe B4.7 in the various mutants.

(A) DNA from various female mutants balanced over FM7 or Basc and homozygous controls and were cut with BamHI, run in a 1% agarose gel and probed with the fragment B4.7 after alkaline transfer (see Materials and Methods). DNA was analysed from females of the following genotypes: 1) xr16/fm7; 2) deb54/Basc; 3) deb154/Basc; 4) deb118/deb118; 5) deb142/deb142; 6) p205C/FM7; 8) p116D/FM7; 9) p205A/4FM7; 11) p[w⁺, ry⁺]E2/P[w⁺, ry⁺]E2; 13) OregonR/OregonR; 4) FM7/FM7 and 15) Basc/Basc.

(B) Diagrammatic representation of the chromosome walk in the 12B region. The EcoRI sites are represented in the upper part, and the BamHI sites in the lower. Important features are the localisation of the P-element insertion P[w⁺, ry⁺]E2, the breakpoint of the deficiency Df(1)LCD and the garnet locus. The 4.7 kb BamHI fragment (B4.7) used as a probe is shown in the lower part of the walk.

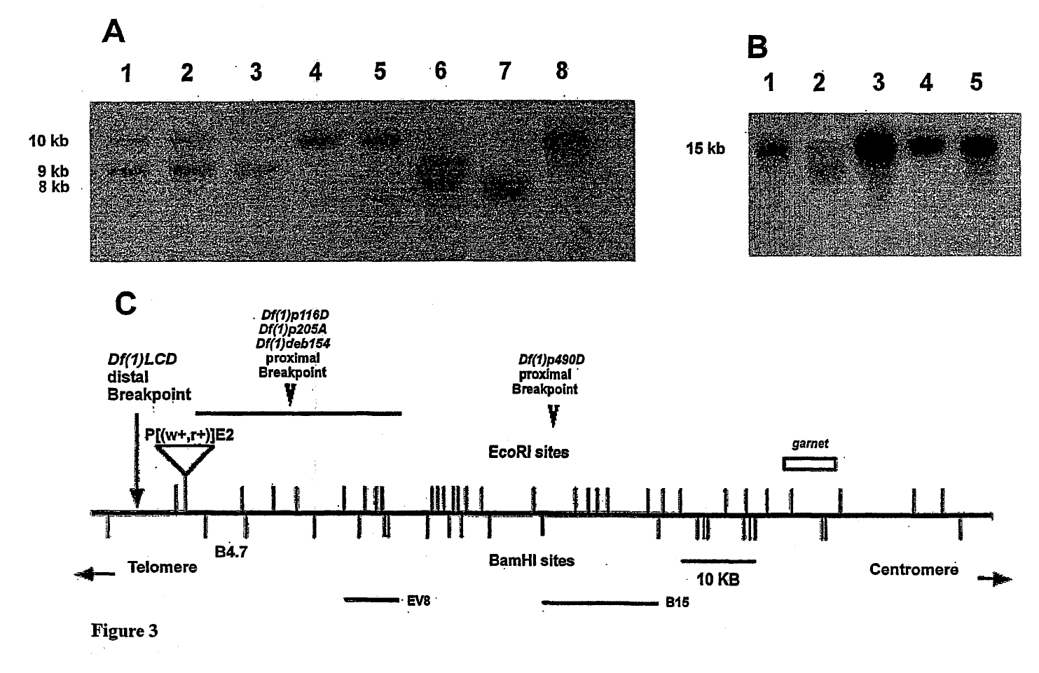

FIG. 3—Molecular characterisation of the 12B region. Reduction of the chromosome walk

(A) DNA from various females mutants balanced over FM7 and homozygous controls were cut with EcoRV, run in a 1% agarose gel and probed with the fragment EV8 after alkaline transfer (see Materials and Methods). DNA was analysed from females of the following genotypes: 1) deb154fm7; 2) p116D/FM7; 3) p205A/FM7; 4) p281A/FM7; 5) p490D/FM7, 6) P[w⁺, ry⁺]E2/P[w+, ry⁺]E2; 7) OregonR/OregonR and 8) FM7/FM7.

(B) DNA from female mutants p281A and p490D balances over FM7 and homozygous controls as presented in (A) were cut with the enzyme BamHI, run in a 1% agarose gel and probed with the fragment B15 after alkaline transfer (see Materials and Methods). DNA was analysed from females of the following phenotypes 1) p281A/FM7; 2) p490D/FM7; 3) P[w⁺, ry⁺]E2/P[w+, ry⁺]E2; 4) OregonR/OregonR and 5) FM7/FM7.

(C) Diagrammatic representation of the chromosome walk in the 12B region. The EcoRI sites are represented in the upper part, and the BamHI sites in the lower. Important features are the localisation of the P-element insertion P[w+, ry⁺]E2, the breakpoint of the deficiency Df(1)LCD and the garnet locus. The 8 kb EcoRV fragment (EV8) and the 15 kb BamHI fragment (B115) used as probes are shown in the lower part of the walk. The localisation of the proximal breakpoint of Df(1)p490D is shown by a vertical arrow in the upper part of the walk. The interval where the proximal breakpoint of the Df(1)p116D, Df(1)p205A and Df(1)deb154 lie is represented by an horizontal line, in the upper part of the map.

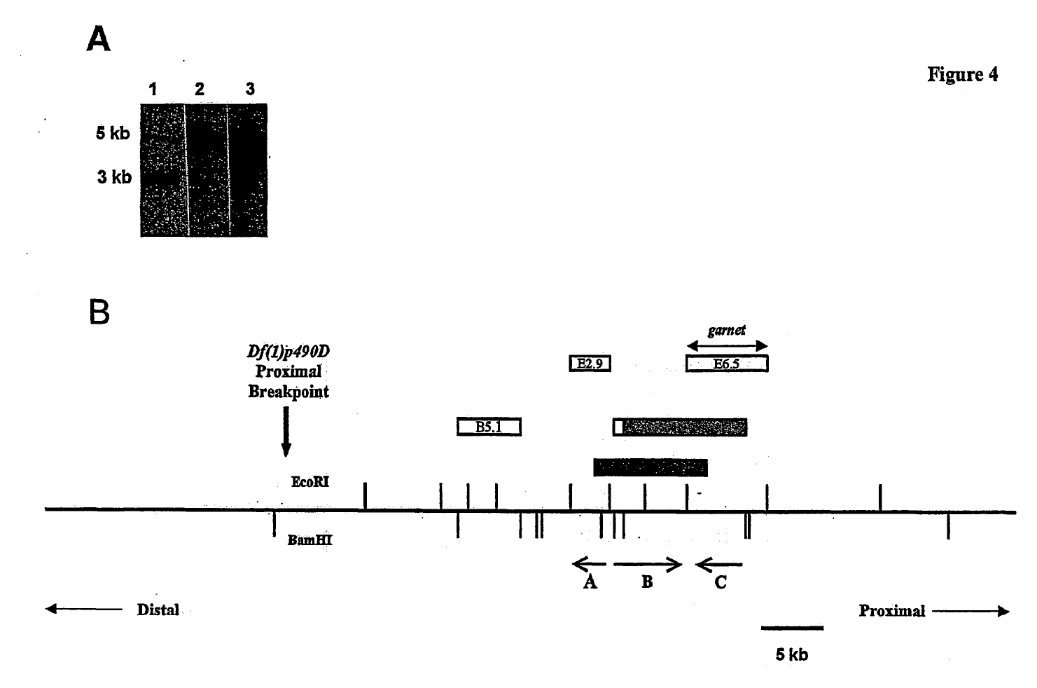

FIG. 4—Diagrammatic representation of the limited region of the walk in 12B to illustrate the position of transcription units.

(A) Northern-blot analysis of 0-4 hour embryos. There genomic probes were used to identify transcription units in the reduced walk: lane 1) E2.9; lane 2) B0.9; and lane 3) B10. Please refer to map in (B) for localisation of the restriction fragments.

(B) The map of the limited walk of region 12B is represented by the central line. The relevant fragments used to search for RFLPs and transcription units are indicated above the map. The B09 fragment is immediately distal to B10, and is represented by an open box. The lines below the map indicate the isolated transcription units. The arrow indicates the direction of the transcription. Important features are the localisation of the proximal breakpoint of df(1)p490D and the garnet locus. The fragments X9 and B10 (grey) were used as mus101 positive and negative rescue constructs respectively.

Symbols: E=EcoRI, B=BamHI, X=Xhol, vertical arrow proximal breakpoint of Df(1)p490D.

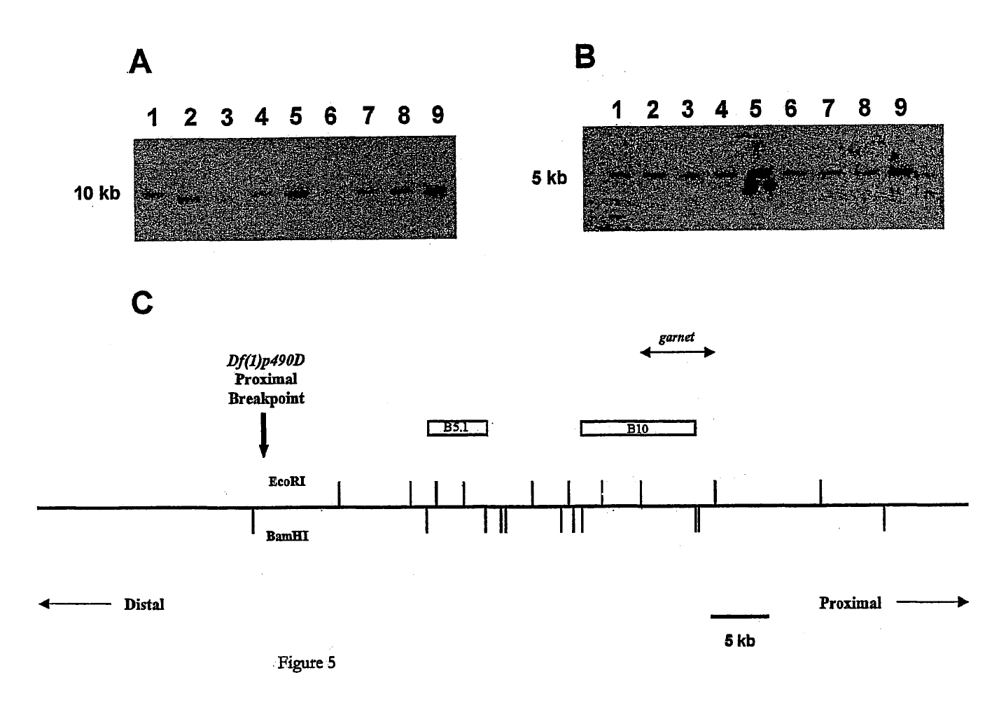

FIG. 5—Identification of RFLPs in three strains of mus101

(A) RFLPs were identified in DNA from three stains of mus101 subjected to Southern-blot following digestion with BamHI. The probe was the B10 fragment shown in (C). Lanes identify DNA from females of the following genotypes: 1) mus101^{k451}/FM3; 2) mus101^{D1}/mus101^{D1}; 3) mus101^{D2}/mus101^{D2} 4) mus101^{tsl}/FM7; 5) mus101^{sm}/FM7; 6) mus101^{lcd}/FM6; 7) FM6/FM6; 8) FM7/FM7 and 9) OregonR/OregonR.

(B) The same blot shown in (A) was probed with the fragment B5.1 showing in (C).

(C) Diagrammatic representation of the reduced chromosome walk, as shown in FIG. 4.1, with special attention to the localisation of fragment B10.

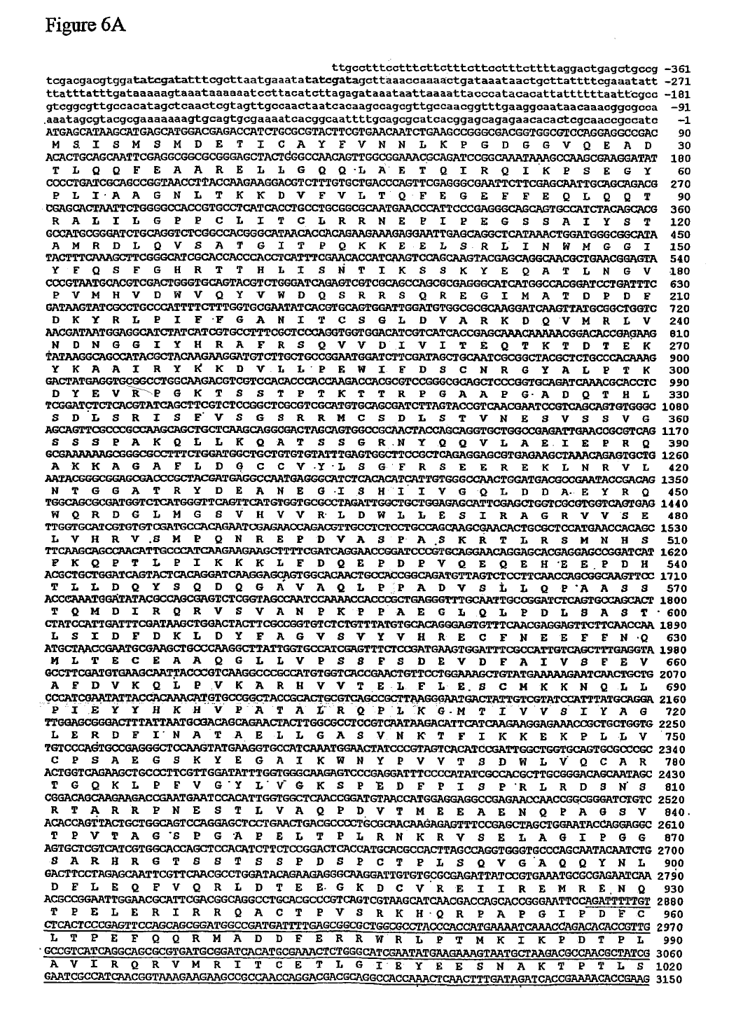

FIG. 6—Genomic sequence of mus101

The genomic sequence of mus101 was obtained using sequence specific primers (see text for details). The non-coding regions (5′ and 3′).are represented by lower case letters. The coding region is represented by upper case letters. Both nucleotide sequence and predicted amino acid sequence are indicated and have different numeration. The potential promoter region has two DRE motifs, represented in blue. The Mus101 predicted protein has seven BRCT domains, represented in green (BRCT I, III, V and VII) and red (BRCT II, IV and VI). The sequence corresponding to cDNAs is underlined. The region where both cDNAs overlap is double underlined. Four polyadenylation signals are represented in bold in the 3′ non-coding region.

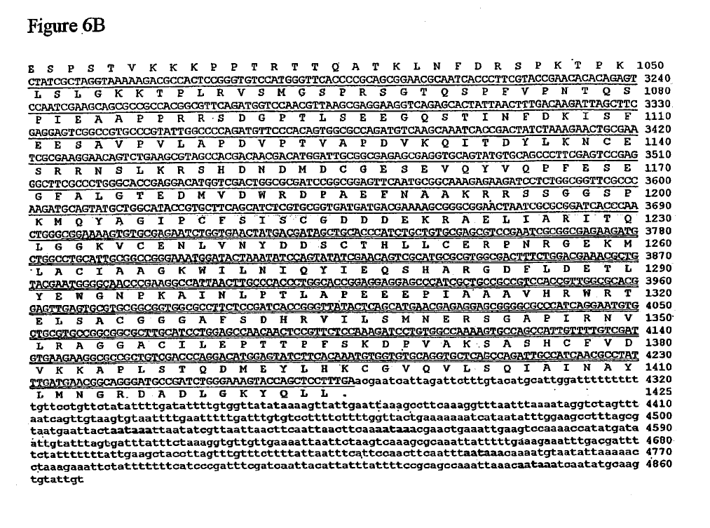

FIG. 7—HCA plot of Mus101 predicted protein.

HCA plot of the Mus101 predicted protein representing the seven BRCT domains. The standard one-letter code for amino acids is used except for proline (★), glycine (♦), serine (□) and threonine (□) (Callebaut et al., 1997). The Mus101 HCA plot was obtained using the program DRAWHCA, available at the URL http://www.lmcp.jussieu.fr/k˜mornon/.

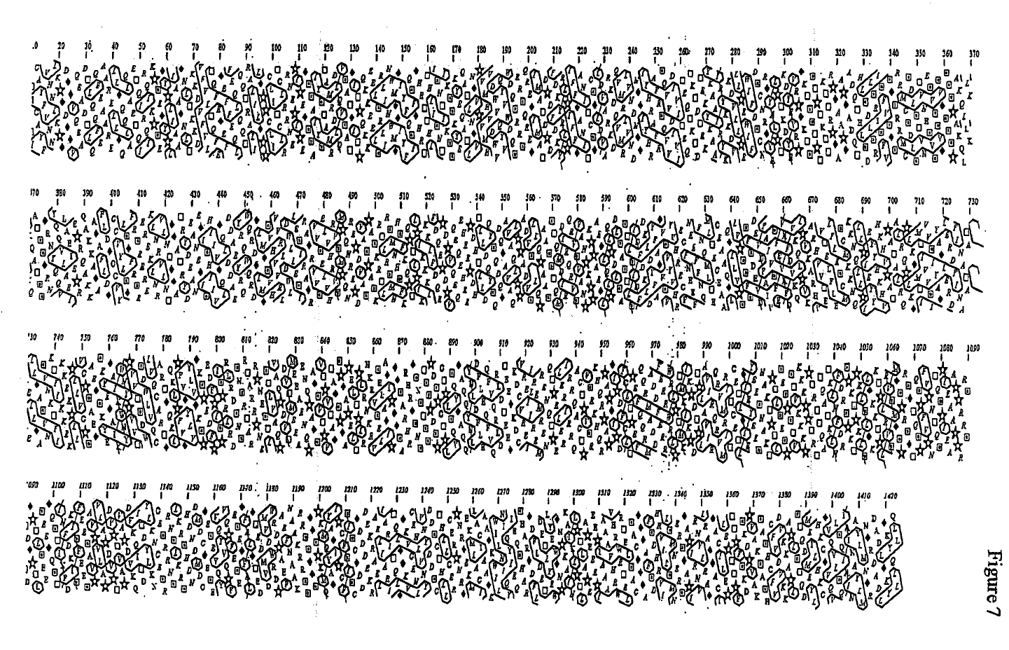

FIG. 8—Comparison of the HCA plots (A) and amino acid sequence (B) of BRCT domain I from Drosophila Mus101, human TopBP1, C elegans clone F37D6.1, fission yeast Rad/4Cut5 and mouse Ect2.

The BRCT domain I was compared between the Drosophila Mus101, human TopBP1, C. elegans clone F37D6.1, fission yeast Rad 4/Cut5 and mouse Ect2 in both HCA plot (A) or in amino acid sequence (B). In the HCA plots, the standard one-letter code for the amino acids is used except for proline (★), glycine (♦), serine (□) and threonine (□) (Callebaut et al., 1997). In “B”, the most conserved amino acids are represented in red, and the conserved amino acids in bold. (C) Schematic representation of the BRCT domains position in Mus101, TopBP1, F37D6.1, Rad4/Cut5 and Ect2. The BRCT domains are represented by blue boxes.

(C) Comparison of the localisation and distribution of BRCT domains (blue boxes) in Mus101, TopBP1, F37D)6.1, Rad4/Cut5 and Ect2.

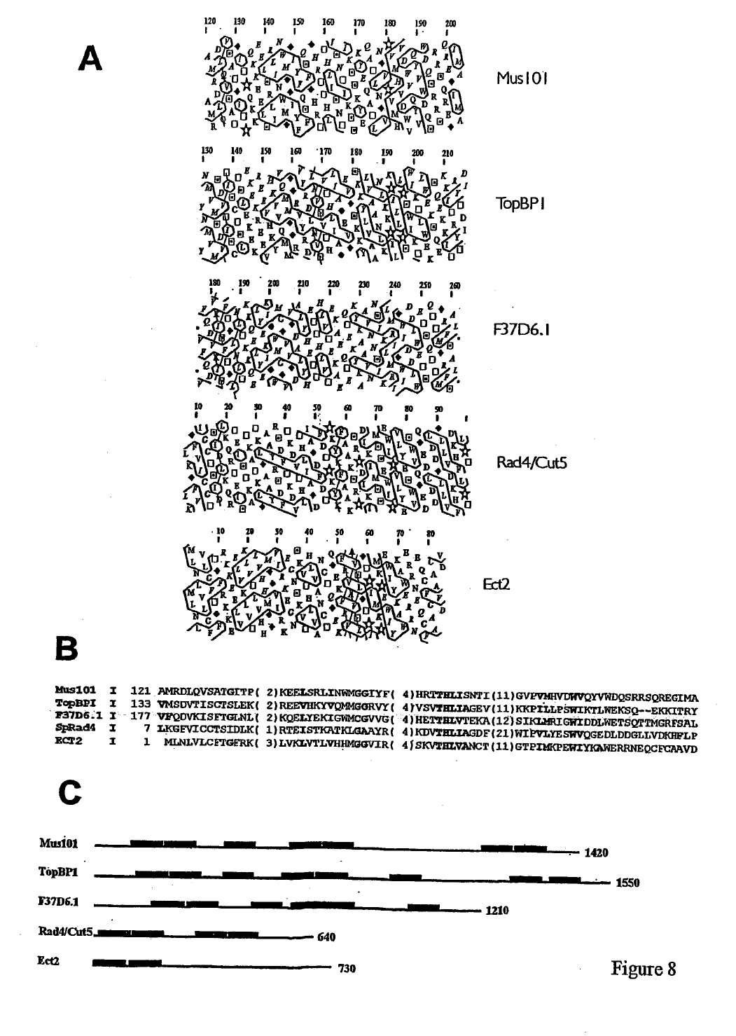

FIG. 9—Pairwise alignment of Drosophila Mus101 predicted protein and human TopBP1.

Alignment of Mus101 predicted protein with human TopBP1 using th. BLAST2 pairwise alignment program.

(A) Alignment of the N-terminal to central region of Mus101 and TopBP1. The scores are: score=228 bits (574), expect=3 e⁻⁵⁸ identity=190/748 (25%), positives=323/748 (42%), gaps=84/748 (11%). The Mus101 BRCT domains I, III and VI are represented in green, and the BRCT domains II and IV in red.

(B) Alignment of the C-terminal region of Mus101 and TopBP1. The scores are: score=102 bits (253), expect e⁻²⁰ identity=67/200 (33%), positives=104/200 (51%), gaps=19/200 (9%). The Mus101 BRCT domain VI is represented in red and domain VII in green.

### EXAMPLES

The first efforts to clone mus101 were carried out in our laboratory (Axton, 1990). First, a more refined cytological localisation of mus101 was achieved by the creation of the Df(1)lcd (12B2-9), allowing mus101 to be placed in the region 12B1,2-12B6. Second, a chromosome walk of 150 kb of genomic DNA was undertaken in this region. Molecular entry into the 12B region was gained using several approaches. The Yolk protein 3 gene (Yp3) was used as a probe to identity cloned genomic segments (as phages and cosmids) from 12B6, the proximal part of the chromosome walk. Microdissection of polytene chromosome bands 12B1,2 and PCR amplification of the DNA recovered provided probes corresponding to the distal part of 12B. Cosmids belonging to contigs 12.2 and 12.6 were joined and their relative orientations established by further chromosome walking (FIG. 1).

The chromosome walk in the 12B region covers 150 kb of cloned genomic DNA. Important landmarks in the walk are the localisation of the breakpoint of the Df(1)lcd in its distal part and the garnet locus, in its proximal part. The Df(1)lcd is the smallest deficiency, that uncovers mus101. mus101 has been mapped by recombination to 0.1 cM to the distal side of garnet (i.e., in the telomeric direction). Thus, with the knowledge of the position of the Df(1)lcd and garnet in the chromosome walk, the area to be searched for mus101 was reduced to approximately 90 kb of chromosome walk.

To reduce the chromosome walk further to a manageable level for the purposes of cloning mus101, we have created new deficiencies in the. 12B region. The different methods employed to achieve this goal are presented in Example 1. The localisation and molecular characterisation of mus101 and the analysis of the predicted Mus101 protein are discussed in Example 2.

Materials and Methods

Drosophila Techniques

Drosophila stocks were maintained on cornmeal medium under standard conditions (Ashburner, 1989; Roberts, 1986). Crosses were performed at 25° C. unless mentioned otherwise. Back-up stocks were maintained at 18° C.

Drosophila Stocks

All Drosophila stocks, balancer chromosomes and phenotypic markers used in this work, unless otherwise stated, are listed in Lindsley and Zimm, 1992 or in the flybase web site (http://morgan.harvard.edu). A list of the most relevant stocks used is presented in the Table 1.

TABLE 1
List of the most relevant Drosophila stocks used in this work.

| Drosophila stock | Reference |
|---|---|
| mus101^{D1} | Boyd et al., 1976 |
| mus101^{D2} | Boyd et al, 1976 |
| mus101^{sm} | A. Schalet, personal communication |
| mus101^{lcd} | Axton, 1990 |
| mus101^{tsl} | Gatti et al., 1983 |
| mus101^{k451} | Perrimon and Gans, 1983 |
| Df(1)LCD(w⁻) | Axton, 1990 |
| P[w⁺, ry⁺]E2 | Levis et al., 1985 |
| y⁺g⁺na⁺Y | A. Schalet, personal communication |

Mutagenesis of Drosophila

X-Ray Mutagenesis

Males aged 3-5 days were irradiated with X-rays to achieve a dose of 40-80 Gy. This dose was obtained by exposing the males for 2, 3 or 4 minutes using the following parameters: 150 kV and 5 mA in a Torrex 150D X-ray machine (Astrophysics Research Corporation). After exposure to the X-rays, the males were transferred to bottles containing double the amount of virgin females (usually 30 males to 60 females). The pairs were left to mate for 2 days, at 25° C. and then transferred twice at two-day intervals to bottles containing fresh food.

DEB Mutagenesis

Males aged 3-5 days were left to starve for 6-10 h in an empty bottle containing filter paper with some drops of water to maintain the humidity. After starvation, the males were transferred to bottles containing filter paper saturated with 5 mM DEB (Diepoxybutane—Sigma) in a 1% sucrose solution. The males were left to feed for an additional 18 h period at room temperature in a chemical fume hood After treatment, the males were transferred to bottles containing fresh maize food and a two-fold excess of Basc virgin females (usually 30 males to 60 females), and kept at 25° C. The pairs were left to mate for 2 days, and transferred twice at two-day intervals to bottles containing fresh food.

All mutagen procedures were undertaken in a chemical fume hood. The glassware and solutions that were in contact with the mutagen were neutralised over night with the neutralisation solution (1 M NaOH, 0.5% thioglycolic acid).

Mutagen Sensitivity Tests

Five pairs of flies of the desired genotype were left to mate in vials containing maize food (vial 1). After two days the adults were transferred to vials containing fresh food (vial 2) and left to mate for two additional days. The, first vial, containing eggs and first instar larvae, were treated with a solution of MMS (Methylmethane sulfonate—Sigma) diluted in distilled water to a desired concentration (usually 0.1%, unless otherwise mentioned). After 24 h in a chemical fume hood, the vials were transferred to 25° C. and left to develop. The progeny of the flies in vial 2 were left untreated and served as a control. All glassware in contact with the mutagen was neutralised as described in section 2.3.2.

DNA Techniques

General Methods

Isolation of DNA from plasmids in small and large scale was done using a Promega Wizard Plus kit (Promega). λ-gt10 cDNA (Poole et al., 1985) was isolated using a Qiagen Lambda mini kit (Qiagen). λZAP EDNA was isolated using the Uni-ZAP™ XR vector (Stratagene). The restriction endonuclease and ligases were purchased from Boehringer Mannheim, and the reactions were performed following the manufacturer's instructions. DNA was subcloned into the vector pBluescript II (Stratagene). Fragments were recovered from an agarose gel using Geneclean II kit (BIO 101). Restriction endonuclease digestion, gel electrophoresis of DNA and blotting of DNA onto nitrocellulose membranes were performed as described by Sambrook et al., 1989.

Genomic DNA was extracted from adult females and total RNA was extracted from Oregon-R wild type embryos according to Ashburner, 1989. Southern and Northern blotting were performed according to Sambrook et al, 1989, using high stringency conditions. DNA fragments were labelled using a Random Primed DNA labelling kit (Moeringer Mannheim). All extractions or reactions made from kits were done following the manufacturer's instructions.

DNA Sequencing

Sequencing reactions were performed using the ABI PRISM big dye terminator cycle sequencing ready reaction kit (Applied Biosystems) following manufacturer's instructions. The reaction were run in an automated 377 Perkin-Elmer sequencer.

Mus101 Primers

Specific primers used to sequence genomic and partial mus101 cDNAs were purchased from Oligosyn, Dundee. The list of primers used is listed in Table 2

TABLE 2

Primers used to sequence the genomic and
partial mus101 cDNAs.

| | |
|---|---|
| | Sequence |
| Name | 5′→3′ |

| | |
|---|---|
| X99P1 | CCGAAGCTATCGCTAGGT |
| X99P3 | AGTCCCACGCGCATGCGA |
| X99P5 | GACGATAGCTGCACCCAT |
| X99P6 | GTGGCGGCGCTGCTTCGA |
| 101C51P1 | GAAGGCGCCGCTGTCGAC |
| B09P1 | CGCACCTCTCGGATCTCT |
| B09P2 | CCACAGCTTCAAGCAGCC |
| E28P1 | AGGCAGGTGATGAGGCAC |
| E28P2 | GTCTTCTTGCTGTCCGGC |
| GENX9P1 | ACCGTTGGCAACGCTGGC |
| GENX9P2 | CGCCATGTGGTCACCGAA |
| GENX9P3 | TGTGTGGTGGTGACCAAC |
| GENX9P4 | CTCTTCGCTTTGGTTTAG |
| GENX9P5 | GCCAGCGTTGCCAACGGT |
| GENX9P6 | TTAGCTTCTCACGCTCCT |
| GENX9P7 | ACGATGATAGATGCCTCC |
| GENX9P8 | TGGCACCAATAAGCCTTG |
| GENX9P9 | GAGCTCCTGAACTGACGC |
| GENX9P10 | TCGCGATTCGCAGTTCTT |
| GENX9P11 | GTCGACAGCGGCGCCTTC |
| GENX9P12 | TAGCGTAATGAATTACTA |

P-Element Mediated Germline Transformation

Flies of the genotype w¹¹¹⁸/w¹¹¹⁸; +/+; 2-3(68C)/+ were collected for 45 minutes at 25° C. on grape juice agar plates. All subsequent operations were performed at 18° C. to delay development of the embryo. Embryos were dechorionated in 3% bleach for 2 minutes, rinsed extensively in tap water and aligned on a cut block of grape juice agar so that the posterior poles protruded over the edge of the block. A slide covered with double side Scotchtape™ was then used to pick up the embryos. Following desiccation for 4 minutes under cold air (hairdryer), embryos were covered in Voltalef 10S halocarbon oil prior to injection for transformation (Karess, 1985). Injection needles (Clarke Electromedical Instruments Ltd) were pulled on a Narishige Scientific Instrument needle puller. Injection of embryos was performed on a Prior micromanipulator using an inverted microscope (Olympus). DNA for injection was centrifuged to remove debris which could block the needle. The constructs were injected at an initial concentration of 0.1-1 mg/ml in injection buffer (5mM KCl, 0.1 mM NaHPO4/Na₂PO₄ pH 7.8) into the posterior pole of the embryo.

Slides were placed in a humid chamber for 48 hours at 18° C. and hatched larvae transferred to vials containing fresh food. Surviving adults were mated individually with w¹¹¹⁸ virgin females or males and progeny showing a w⁺ phenotype were identified as transformants. The chromosome on which the P-element had been inserted was identified by crossing virgins balanced for the second (w¹¹¹⁸/w¹¹¹⁸; Tft/CyO; +/+) or third chromosomes (w¹¹¹⁸/w¹¹¹⁸; +/+; TM3/TM6c) to w⁺ transformant males. All transformants were kept as homozygous stocks. Table 2.4 shows the transformants obtained from injection of the positive and negative constructs.

The X9 fragment, a 9 kb XhoI fragment from cosmid 44F7 was subcloned into the XhoI site of the transformation vector pW8 (Klemenz et al., 1987) to form the positive rescue construct p[w⁺, X9]. The B10 fragment, a 10 kb BamHI fragment from cosmid 22F12 was subcloned into the BamHI site of the transformation vector pCaSpeR (Pirrota, 1988) to form the negative rescue construct p[w⁺, B10].

TABLE 3
List of transformant lines obtained after injection of mus101
positive and negative rescue constructs. The chromosome
in which the P-element has been inserted is indicated.

| Construct name | Chromosome | Construct |
|---|---|---|
| p[w⁺, X9]C9A | 3rd | + |
| p[w⁺, X9]C11A | X | + |
| p[w⁺, X9]D3B | 3rd | + |
| p[w⁺, X9]D13D | 2nd | + |
| p[w⁺, X9]H5 | 2nd | + |
| p[w⁺, X9]H5C | 3rd | + |
| p[w⁺, X9]J10B | 2nd | + |
| p[w⁺, X9]J14A | 2nd | + |
| p[w⁺, X9]K8A | 2nd | + |
| p[w⁺, X9]K8B | X | + |
| p[w⁺, X9]L4B | 3rd | + |
| p[w⁺, X9]M1 | 2nd | + |
| p[w⁺, X9]M5B | 2nd | + |
| p[w⁺, X9]M6 | X | + |
| p[w⁺, B10]A4D | 3rd | − |
| p[w⁺, B10]A8A | 3rd | − |
| p[w⁺, B10]A8E | 2nd | − |
| p[w⁺, B10]E10A | 3rd | − |
| p[w⁺, B10]F4A | X | − |
| p[w⁺, B10]F4B | 3rd | − |
| p[w⁺, B10]F7A | 2nd | − |
| p[w⁺, B10]F10E | 3rd | − |
| p[w⁺, B10]F11A | X | − |
| p[w⁺, B10]I1A | X | − |
| p[w⁺, B10]J3C | 2nd | − |
| p[w⁺, B10]J4A | 2nd | − |
| p[w⁺, B10]J5B | 3rd | − |

### Example 1

Creation of New Deficiencies in the Region 12B1,2-6

At the onset of this work, we were faced with the problem of correlating the existing cytogenetic maps that defined the position of mus101 with the 150 kb chromosome walk that had been carried out in the region 12B (Axton, 1990). It was imperative to create new deficiencies in this area not only because these might create new alleles of mus101, but because they might identify part of the chromosome walk not containing the two genes. It would be desirable to create small deficiencies in order to reduce the walk to a manageable size.

Three approaches were used to create deficiencies in the region 12B: X-ray mutagenesis; chemical mutagenesis using DEB; and imprecise excision of P-elements of which the latter generated useful deficiences. The imprecise excision of P-elements is a powerful means of mutagenesis. When a P-element excises from the chromosome, three events can occur: the excision can be precise, imprecise or it can cause internal rearrangements. We were aiming to obtain imprecise excision of the P-element in which simultaneous loss of adjacent DNA would occur. Such deletions extend from the position of the insertion of the P-element in the chromosome.

Independent of the source of mutagen used, it is necessary to use a chromosome carrying a phenotypic marker in a region in the vicinity of the region to be mutated. In the case of this work, I have used the strain P[w⁺, ry⁺]E2 (Levis et al., 1985).

Strain P[w⁺, rv⁺]E2

The Drosophila strain P[w⁺, ry⁺/E2 cares a P-element in the region 12B1-2 (Levis et al.,1985) This P-element has white (w⁺) and rosy (ry⁺) phenotypic eye color markers. The presence of the w⁺ marker facilitates the analysis of mutated chromosomes, since w mutant and wild-type eye colour can be easily recognised.

The P-element in the P[w⁺, ry⁺]E2 strain had been localised internally in the 12B region of the chromosome walk. It lies in the distal part of the walk within a 12 kb BamHI fragment found in wild-type (Oregon-R or Cantons-S) DNA. It is detected using a 4.7 kb BamHI fragment from the end of the insert in the cosmid 29E9 (named B4.7). In the strain P[w⁺, ry⁺]E2, this probe recognises a 6 kb BamHI fragment that extends from the internal BamHI site in the P-element to a site in the proximal flanking DNA (FIG. 2B).

Knowledge of the P-element location at molecular level was essential to analyse the new chromosomal rearrangements generated by mutagenesis. Specifically it facilitates the determination of whether a mutated chromosome has a deletion internally in the region of the walk or not. If the 6 kb BamHI band is present, it means that the chromosome P[w⁺, ry⁺]E2 is still intact in this region. However, if it is absent or altered, a small deletion (or rearrangement) has occurred.

P-Element Imprecise Excision

The generation of P-element imprecise excision mutants is a very direct means of mutagenesis. When a P-element excises from its original site, it can carry segments of the chromosome. The amount of DNA deleted and the frequency of this event depends upon the P-element's insertion site. Deficiencies were created in the region 12B by P-element mobilisation. The extent of the deletions were monitored both genetically and by molecular approaches. To this end, males containing the P-element were generated and a transposase source from whose progeny lethal strains were selected with loss of w⁺. We chose to look for lethal mutants, since mus101 has lethal phenotypes. We searched for mutations that would delete one, other, or both of these genes. However, viable mutations with loss of w⁺ can still be generated that delete part of the chromosome walk. These would not be detected, but in any event the molecular analysis of such mutants would be very time consuming and they might prove not to be informative.

The scheme of the crosses is illustrated below. In the first cross, made en masse, homozygous females carrying the P-element P[w⁺, ry⁺]E2 are crossed with males carrying the transposase, to generate “jump-start males” in the next generation. Three “jump-start males” were crossed with five crm/FM7 virgin females (a stock carrying the lethal mutation cramped (crm), affecting a polycomb group gene in the region 3C Yamamoto et al., 1997 was used as a source of the FM7 balancer chromosome). This cross not only balanced the progeny, but also allowed us to select flies where the transposition event had occurred by loss of the w⁺ marker. The third cross was designed to check lethality. Four P[w⁺, ry⁺]E2*/FM7 females from each G₁ vial were crossed individually to FM7 males. The lines in which homozygous males were not recovered were kept balanced over FM7 for further genetic analysis.

A total of 1,698 G₂ crosses were analysed, and 23 lethal mutants were recovered.

$${G}_{0} : P [ {w}^{+} , {ry}^{+} ] E2 \text{/} P [ {w}^{+} , {ry}^{+} ] E2 ( 60 ) ⊗ + \text{/} Y ; Δ2 \text{-} 3 , Sb \text{/} Df ( 30 )$$

$${G}_{1} : crm / FM7 ( 5 ) ⊗ P [ {w}^{+} , {ry}^{+} ] E2 \text{/} Y ; Δ2 \text{-} 3 , Sb \text{/} + ( 3 )$$

$${G}_{2} : P [ {w}^{+} , {ry}^{+} ] {E2}^{*} \text{/} FM7 ( 1 ) ; + \text{/} + ⊗ FM7 \text{/} Y ( 3 )$$

$$Where {(}^{*} ) means loss of {w}^{+} . \\ Select : lethal mutants , discard all viable ones . \\ Numbers in brackets represent the average number of flies used per bottle \text{/} vial .$$

Genetic Analysis of P-Imprecise Excision Mutants

The mutants generated by P-element imprecise excision were analysed in complementation tests with strains mus101^{sm} and dd4^{08.20}. Virgin p/FM7 females were crossed with mus101^{sm}/y⁺g⁺na⁺Y and dd4^{08.20}/y⁺g⁺na⁺Y. The recovery (+) or not (−) of the heterozygous combinations is presented in Table 4. These mutants can be divided into two classes: those that complement both mutations tested (p44C, p71A, p100B, p116D, p205A, p205C, p222C, p281A, p352D, p426D and p490D), and those that fail to complement both (p30A, p69A, p189C, p232B, p263C, p244B, p259B, p309B, p327D, p333C, p414C and p514C).

In order to test if the P mutations that delete the gene mus101 would also delete garnet, an eye colour mutation localised in the proximal part of the walk (FIG. 2B), complementation tests were performed using the garnet strain wy²g⁴. The progeny of these crosses were checked for the presence of garnet (g⁻) or wild type (g⁺) eye colour. The results are given in Table 4.

All P-element imprecise excision mutants that delete the gene mus101 delete garnet as well. This result indicates that the deletions extend from the distal part of the walk to at least, or beyond the garnet locus. The mus101 locus was mapped by recombination by A. Schalet (Yale University) to about 0.1cM to the distal side of garnet (i.e., in the telomeric direction). Unfortunately these deletions are not informative with respect to mapping the mus101 locus, since they remove a great part of the walk.

Five strains that complement the mus101 mutations were also tested for their ability to complement garnet. The strains p116D, p205A, p281A, p426D and p490D when heterozygous with garnet have wild-type eye colour (Table 4).

Molecular Analysis of P-Element Imprecise Excision Mutants

The eleven mutants that complement the mus101 mutation were analysed at the, molecular level in Southern-blot experiments. DNA from p/FM7 females was cleaved with BamHI and probed with the genomic fragment, B4.7 referred to in section 3.1. The hybridisation patterns of these mutants fall in 2 classes:

a) No, deletion of the chromosome walk: This class includes mutants p44C, p71A, p110B, p205C, p352D and p426D. Southern-blots of these mutants reveal bands of 6 kb and 12 kb, corresponding to the P[w⁺, ry⁺]E2 and FM7 chromosomes respectively (see mutant p205C as an example, in FIG. 2A, lane 6). One case of chromosome rearrangement is seen in mutant p222C (FIG. 2A, lane 7), which has a 8 kb band instead of a 6 kb derived from the chromosome P[w⁺, ry⁺]E2. In these mutants there is not a deletion of the internal part of the walk. There might be, however, a deletion in the other side of the P-element, causing the loss of the white eye colour and the lethal phenotype. It is also possible that the loss of eye colour is due to an internal rearrangement in the P-element, and the lethality is due to a second site mutation. These mutants were not further characterised.

b) Deletion of the chromosome walk: In this class of mutants, represented by strains p116D, p205A, p281A and p490D, the 6 kb band present in the strain P[w⁺,ry⁺]E2 was deleted (FIG. 2A lanes 8-11). The DNA of these mutants was further analysed to determine the extend of the deletion in the walk (see below).

Of the 23 w⁻ lethal mutations obtained by P-element imprecise excision, 4 had a deletion in the walk. It is known, by, genetic analysis, that these 4 mutants (p116D, p205A, p281A and p490D) complement the lethal phenotypes of mutant mus101^{sm}. Furthermore, these mutant chromosomes, when heterozygous with mutant wy² g⁴ have wild-type eye colour, indicating that the garnet locus localised in the proximal part of the walk is still intact. It thus can be concluded that these deficiencies extend proximally to a point between the P-element and the garnet locus. The precise localisation of these breakpoints would therefore be of great importance to exclude part of the walk that does not contain the mus101 gene.

TABLE 4
Complementation analysis of P imprecise excision mutants. p/FM7
females were crossed with mus101^{sm}/y⁺g⁺na⁺Y, dd4^{08.20}/
y⁺g⁺na⁺Y and wy²g⁴/Y.

| P imprecise | | | |
|---|---|---|---|
| mutant | mus101^{sm} | wy²g⁴ | y⁺g⁺na⁺Y |
| p30A | − | − | − |
| p44C | + | NT | − |
| p69A | − | − | − |
| p71A | + | NT | − |
| p100B | + | NT | − |
| p116D | + | + | − |
| p189C | − | − | − |
| p205A | + | + | − |
| p205C | + | NT | + |
| p222C | + | NT | − |
| p232B | − | − | − |
| p259B | − | − | − |
| p263C | − | − | − |
| p264B | − | − | − |
| p281A | + | + | − |
| p309B | − | − | − |
| p327D | − | − | − |
| p333C | − | − | − |
| p352D | + | NT | − |
| p414C | − | − | − |
| p426D | + | + | − |
| p490D | + | + | − |
| p514C | − | − | − |

(+) represents complementation of the phenotype, (−) non-complementation and (NT) not tested.

Reduction of the Chromosome Walk

In the previous sections we have presented genetic and molecular data concerning the creation of new deficiencies in the 12B region. Of all mutants recovered (54 in total) using different methods, only five have deleted part of the walk. Among these mutants are the DEB-induced mutant deb154 and the P-element imprecise excision mutants p116D, p250A, p281 and p490D. The following section presents data about the extent of these deletions in the chromosome region represented by the walk.

Genetic analysis performed earlier (see above) has shown that these mutants complement the mus101 mutation. Thus it could be anticipated that the molecular information I would obtain would delimit the chromosome region containing the mus101 gene in relation to the original P-insertion site of P[w⁺,ry⁺]E2.

The strategy used to determine the breakpoint of the deficiencies in the mutants deb154, p116D, p205A, p281A and p490D was as follows: DNA was extracted from mutants balanced over FM7, cleaved with different restriction endonucleases and analysed in Southern-blotting experiments probed with several fragments from the genomic walk.

The first informative genomic, region was an 8 kb EcoRV fragment recovered from cosmid 110D5—referred to as RV8 (FIG. 3C). This is a polymorphic region between the strains P[w⁺, ry⁺]E2, wild-type (OregonR) and FM7 balancer chromosomes. The sizes of fragments that this probe recognises in each one of these strains are: 9 kb, 8 kb and 10 kb, respectively when DNA is cleaved with the enzyme EcoRV (FIG. 3A, lanes 6, 7 and 8).

DNA from the mutants deb154, p116D, p205A, p281A and p490D and appropriate controls was cleaved with the enzyme EcoRV, blotted and probed with the fragment EV8. The mutants deb]154, p116D and p205A show, after hybridisation, the 9 and 10 kb bands corresponding to the strain P[w⁺, ry⁺]E2 and to the FM7 chromosome (FIG. 3A, lanes 1-3). The mutants p281A and p490D show only the 10 kb band, corresponding to the FM7 chromosome (FIG. 3A, lanes 4 and 5).

From this analysis it can be concluded that the breakpoint of deficiencies deb154, p116D and p205A lie in the interval between the site of the P-element insertion and the location of the fragment EV8 (FIG. 3C). The precise location of these breakpoints were not determined. The deficiencies in strains p281A and p490D span the area covered by the fragment EV8.

A second informative genomic fragment was a 15 kb BamHI fragment from cosmid 165E3, referred as B15 (FIG. 3C). DNA was extracted from mutant females over the FM7 balancer and from controls, cut with BamHI, Southern-blotted and probed with the fragment B15.

DNA from the P[w⁺, ry⁺]E2, OregonR and FM7 chromosomes shows a 15 kb band after hybridisation, as does DNA from the mutant p281A (FIG. 3B, lanes 3, 4, 5, and 1, respectively). DNA from mutant p490D shows an additional 13 kb band, indicating the location of the breakpoint for this mutant (FIG. 3B, lane 2). The breakpoint of the deficiency p490D (Df(1)p490D) is shown by a vertical arrow in FIG. 3C. It is not clear from this blot if the mutant p281A has a deletion that extents into the B15 region, or if the breakpoint lies in between the EV8, and B15 region. No further characterisation was done for the mutant p281A.

The results presented in this section effectively reduce the molecular limits of the chromosome region that contains the mus101 gene. The region that extends from the P-element in the proximal part of the walk to the region covered by the genomic fragment B15 does not contain the mus101 gene. Therefore, the gene should be located towards the proximal part of the walk from B15.

### SUMMARY

In this example, we have shown the data obtained after mutagenesis of the chromosome P[w⁺, ry⁺]E2 (Levis et al., 1985) using imprecise excision of P-elements. The aim of using this technique was to recover small deficiencies in the 12B region, in order to eliminate DNA represented by segments of the walk area as candidate regions for the molecular localisation of the mus101gene.

We have recovered 26,458 DEB-treated chromosomes, 10 of which were w⁻. Only one of these mutants, deb154 has a deletion in the 12B walk area, Even though the exact molecular localisation of the breakpoint was not determined, it may be deduced to lie between the P-element insertion point in the walk and the 8 kb EcoRV fragment represented in FIG. 3C. Thus, the deletion in the mutant deb154 is not bigger than 25 kb.

The mutagenesis using imprecise excision of the P-element proved to be very effective. 23 lethal mutations were obtained in a total of 1,698 w mutants (1.35%), and four lethal mutants have a deletion in the region of the walk. The Df(1)p490D contained the biggest deletion (see section 3.5); the molecular localisation of the proximal breakpoint of this deletion has lead to a significant reduction in the walk from 50 to 60 kb, starting from the P-element.

Furthermore, genetic analysis showed that the Df(1)p490D complements lethal mutations of mus101. So, the gene of interest is not localised within this 50-60 kb of the walk.

The distance between the proximal breakpoint in Df(1)p490D and the garnet locus is approximately 30 kb. Recombination studies by A. Schalet (Yale University) have positioned mus101 to about 0.1 cM to the distal side of garnet. Thus, by both molecular and genetic analyses, one can say that mus101 is located in this 30 kb interval. This is indeed the case, and the data concerning the molecular cloning of mus101 will be presented in the following chapter.

### Example 2

Cloning of mus101

In the previous chapter data has been presented showing the strategy used to limit the region of the chromosome walk in the region 12B that should encompass the mus101 locus. A new deficiency, in the 12B region, named Df(1)p490D, created by P-element imprecise excision has deleted chromosomal DNA represented by approximately 60 kb of the original chromosome walk. The Df(1)p490D complements all phenotypes of mus101, therefore the mus101 locus is outside the region uncovered by this deficiency. However, localisation of the proximal breakpoint of Df(1)p490D, together with genetic mapping of mus101 distal to garnet, has resulted in a reduction of the original walk that should contain mus101 to within a 30 kb interval.

In this example we will present the strategy used to localise precisely the mus101 gene in mutant alleles utilising restriction fragment length polymorphisms (RFLPs). Partial cDNAs were isolated from different libraries, and the sequence of both genomic DNA and partial cDNAs for mus101 were determined. Upon P-element mediated germline transformation I confirmed that the cloned transcription unit can rescue all mutant phenotypes of mus101.

Localisation of mus101

We first wished to determine whether any of the mus101 alleles showed an RFLP in the region extending from the proximal breakpoint of the Df(1)p490D to the gene garnet. This area corresponds to approximately 30 kb of cloned DNA, and is diagrammatically represented in FIG. 4B. Genomic fragments from the distal-most part of this region did not detect RFLPs when used as probes in Southern-blots of restriction endonuclease digested DNA extracted from various mus101 alleles. The most distal fragment tested in these experiments was a 5.1 kb BamHI fragment (B5.1, FIG. 4B). Thus if the distal part of the 30 kb region to the point of this 5.1 kb fragment (B5.1) can be eliminated, the new area to be searched for RFLPs in mus101 mutants is further reduced to approximately 15 kb.

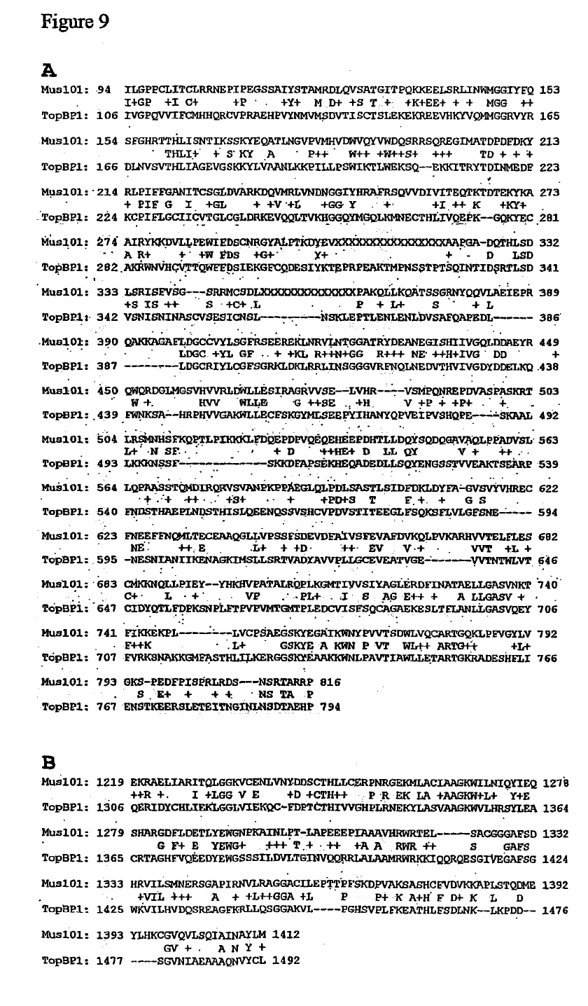

We have tested all mus101 alleles for RFLPs in this remaining 15 kb of the reduced chromosome walk. We extracted DNA from homozygous females (mus101^{D1}, mus101^{D2}), or females heterozygous with a balancer chromosome (mus101^{lcd}/FM6, mus101^{sm}/FM7, mus101^{tsl}/FM7 and mus101^{K451}/FM3) for cleavage with BamHI. After electrophoresis and transfer, the membrane was probed with genomic fragments from the proximal part of the region. The fragment B10 (FIG. 5C) detects a RFLP in the lethal strains mus101^{lcd} and in the mutagen-sensitive strains mus101^{D1} and mus101^{D2} (FIG. 5A, lanes 2 and 3). In balancer chromosome (FM6 or FM7) or wild type (Oregon-R), this probe detects a fragment of 10 kb (FIG. 5A, lanes 7-9). In the strain mus101^{lcd} the B10 fragment reveals two bands: the 10 kb of the balancer chromosome FM6, and a second band, of approximately 12 kb, corresponding to the mus101^{lcd} chromosome (FIG. 5A, lane 6). In the strains mus101^{D1} and mus101^{D2} the fragment B10 identifies only one band, of approximately 9 and 8.5 kb respectively (FIG. 5A, lanes 2 and 3). To determine if the 12 kb band present in the strain mus101^{lcd} is not due to partial cleavage of DNA, the blot was re-probed with the fragment B5.1 (FIG. 5C). As can be observed in FIG. 42B, the probe identifies a band of approximately 5 kb in all lanes.

The identification of RFLPs in three independent mus101 alleles in a region corresponding to the B10 restriction fragment strongly suggests that mus101 locus is located at this site. The B10 fragment partially overlaps with a 6.5 kb EcoRI fragment (E6.5, FIG. 4B), known to contain the garnet locus suggesting that mus101 and garnet are closely physically linked, consistent with previous recombination mapping data.

Identification of Transcription Units in the mus101 Area

Two methods were used to identify transcription units in the area surrounding mus101: Northern-blots and isolation of cDNAs.

Northern-Blot Analysis

Total RNA extracted from 0-4 hour embryos was used in the Northern-blot experiments. The blots were probed with genomic fragments E2.9, B0.9 and B10 (FIG. 4B). These are adjacent fragments in the walk and cover an area of about 14 kb. The fragments B0.9 and E2.9 are distal to B10.

The fragment E2.9. recognised a transcription unit of approximately 3 kb (FIG. 4A, lane 1 and FIG. 4B, transcript A). The transcription unit A is not likely to be mus101, since it is not detected by fragment B10. The fragment B10 identified two transcription units, one of approximately 5 kb and one of approximately 3.5 kb (FIG. 4A, lane 3, FIG. 4B, transcripts B and C, respectively). The fragment B0.9, distal to B10, also recognised the transcription unit B (FIG. 4A, lane 2, FIG. 4B), indicating that it spans the B0.9 and B10 restriction fragments. The transcription unit C is likely to be garnet, which is known to lie within the B10 fragment and which corresponds in size to the embryonic garnet transcription unit. It is therefore likely that transcription unit B corresponds to mus101, since it is detected by fragment B10, the fragment that shows RFLPs in the strains mus101^{lcd}, mus101^{D1} and mus101^{D2}.

Screening of an Embryonic λgt10 cDNA Library

We have screened the 0-3 hour embryonic library constructed in the vector λgt10 (Poole et al., 1985) for mus101 cDNAs. As a probe I used a 9 kb XhoI fragment (X9) which partially overlaps the fragments E2.9, B10 and E6.5 and contains the whole B0.9 fragment (FIG. 4B). With this fragment we would be able to confirm the previous identified transcription units reported above.

Six clones were isolated (X91, X93, X94, X95, X96 and X99) and proved to have inserted EcoRI fragments that varied from 1.2 to 3 kb. In order to facilitate the molecular analysis of these cDNAs, they were sub-cloned into the EcoRI site of the vector pbluescript (Stratagene), as described in the Materials and Methods.

The sequence of both ends of the six cDNAs was determined using the commercially available primers, as described in the Materials and Methods. Analysis of the partial DNA sequence revealed that the cDNAs X91, X94, X95 and X96 were overlapping, and they correspond to the sequence of the garnet cDNA. Thus, these four cDNAs represent transcription unit C (garnet) in FIG. 4B.

The partial sequence of cDNAs X93 and X99 are non-overlapping, and do not correspond to the garnet cDNA. The sequence of the cDNA-X93 does overlap that of a cDNA previously isolated using the fragment E2.9 as probe (data not shown), and is likely to be transcription unit A (FIG. 4B). A homology search using the BLASTX program (Altschul et al., 1997) revealed that the partial sequence of the cDNA X93 is similar to the human clone KIAA0544 (Nagase et al., 1998, Genbank accession number 3043612) and to the C. elegans locus CEC11H1 (Genbank accession number Z70205), identified in the genome project. Although no function has been assigned to these loci, the human KIAA0544 has significant similarity to human cell growth regulators and apoptosis inhibitors (Nagase et al., 1998).

A BLASTX search using the clone X99 shows similarity with a human cDNA Pagase et al., 1996), which in turn is similar to the S. pombe rad4⁺/cut5⁺ gene (Fenech et al., 1991; Saka and Yanagida, 1993). Mutants for rad4⁺/cut5⁺ show sensitivity to a variety of DNA damaging agents, and it has been shown that the product of the gene rad4⁺/cut5⁺ is essential for S phase, also being important in the replication checkpoint. Due to similar phenotypes presented by mus101 mutants and rad4⁺/cut5⁺ mutants of S. pombe, and the similarities in sequence, the cDNA X99 is a good candidate for mus101. So, the cDNA X99 was renamed to mus101B1. The genomic region encoding this transcript was subsequently shown to rescue mus101. Further discussion concerning the sequence homology will be addressed below.

The cDNA mus101B1 was sequenced in both directions using oligonucleotide primers designed specifically for its sequence (Materials and Methods). The total length of the mus101B1 cDNA is 1372 bp, and it has an open reading frame (ORF) of 456 amino acids (FIG. 6, underlined). The cDNA mus101B1 is not full length and does not contain the 3′ non-coding region. It corresponds to nucleotides 2870 to 4241 of the mus101 genomic sequence (see below). There is a difference of one nucleotide between the cDNA and genomic sequences at position 4227 (genomic number). In this position there is a cytosine in the genomic and an adenine in the cDNA sequence. This nucleotide substitution is in the third position of the codon, and does not lead to an amino acid change (in both cases the triplet codes for an alanine). This nucleotide substitution must be due to natural polymorphism present in the different wild-type strains used to construct the genomic and cDNA library.

The expected size of a complete mus101 cDNA is about 4.5 to 5 kb (as judged by the size of transcript B identified by Northern-blot). Since the cDNA mus101B1 is much smaller than the predicted size, We conclude it only represents a partial reverse transcript. The search for a complete cDNA was therefore necessary.

Screening of Embryonic λZAP cDNA Library

In order to isolate a complete mus101 cDNA, We screened a λZAP 2-14 hour embryonic cDNA library (Stratagene), using the cDNA mus101B1 as a probe, as described in Materials and Methods. Only one cDNA was recovered, and named mus101B2.

The cDNA mus101B2 was totally sequenced using oligonucleotide primers specific for its sequence. Both strands were sequenced, as described in Materials and Methods. It has an ORF of 193 amino acids and 548 bp of 3′ non-coding region, including four consensus polyadenylation signals (AATAAA—FIG. 6, bold). The cDNA mus101B2 corresponds to nucleotides 3966 to 4868 of the genomic sequence presented in FIG. 6, underlined As with cDNA mus101B1, the mus101B2 cDNA also differs from the genomic region in position 4227.

There is a 545 bp overlap between the 3′ end of mus101B1 cDNA and the 5′ end of mus101B2 cDNA (FIG. 6, double underlined). Thus the cDNA mus101B2 is not complete, and only represents an extension to the 3′ end of the mus101B1 cDNA. The cDNA mus101B2 is underlined in FIG. 6, as a continuation of cDNA mus101B1.

The low number of cDNAs recovered in two independent embryonic libraries, suggests that mus101 is expressed at low levels during this stage of development. A search for a complete cDNA in libraries representing other stages of the fly development is necessary.

Identification of mus101 Locus by P-element Mediated Germ-Line Transformation

In order to confirm the cloning of mus101, two genomic constructs were designed for use in P-element mediated germ-line transformation experiments: one expected to be the positive, containing the whole mus101 sequence and promoter region, and a negative control, lacking the 5′ region of mus101 (coding and non-coding regions).

The genomic fragment X9, a 9 kb XhoI fragment referred to in section 4.2.2 (FIG. 4B) was used as a positive construct. The X9 fragment was cloned in the transformation vector pW8 to form plasmid p[w⁺, X9] (Materials and Methods). This fragment contains the complete mus101 gene and incomplete sequences of transcription unit “A” and garnet. The B10 fragment (FIG. 4B) was subcloned in the transformation vector pCasPeR, to give the negative construct p[w⁺, B10] (Materials and Methods). The fragment B10 is missing approximately 2 kb from the 5′ end of mus101.

Both constructs were injected into w¹¹¹⁸; Δ2-3(68C)/+ embryos as described in the Materials and Methods. Fourteen independent transformant lines were recovered for the positive construct p[w⁺, X9]; three on the X-chromosome, seven on the second chromosome and four on the third chromosome. Thirteen independent transformant lines were recovered for the negative construct p[w+, B10]; three on the X-chromosome, four on the second and 6 on the third chromosome. These lines were maintained as homozygous stocks (see Materials and Methods).

To test whether these constructs were able to rescue the phenotypes of several mus101 alleles, complementation tests were performed. Homozygous mus101^{D1} and mus101^{D2} and heterozygous mus101^{lcd}/FM6, mus101^{sm}/FM7c, mus101^{tsl}/FM7c and mus101^{K451}/FM3 virgin females were crossed with transformant males in which the P-element has been inserted in the autosomes (11 independent lines for positive construct p[w⁺, X9] and 10 independent lines for the negative construct p[w⁺, B10]). The mutagen-sensitivity test was performed using a dose of 0.1% MMS, as described in Materials and Methods. In this complementation test the--phenotype analysed was lethality of hemizygous males after mutagen treatment. The permissive temperature of mus101^{tsl} is 17° C. To test the rescue of the temperature sensitive phenotype, all crosses were done at 25° C. The rescue of the mus101 phenotype was monitored by the recovery of hemizygous males with w⁺ eye colour.

As can be observed in Table 5, the construct p[w⁺, X9] rescues the phenotypes of all mus101 alleles tested, except for the lethality in the allele mus101^{lcd}. In contrast, the construct p[w⁺, B10] rescues none of the various mus101 mutant phenotypes. The positive construct p[w⁺, X9] does not rescue the lethal phenotype of the allele mus101^{lcd} due to a lethal second site mutation, localised outside the duplication y⁺g⁺na⁺Y (12A8; 12F).

It can be concluded that the mus101 gene corresponds to the ORF of the transcription unit B which is initiated in fragment X9, and disrupted in fragment B10.

TABLE 5
Positive p[w⁺, X9] and negative p[w⁺, B10] rescue constructs of mus101
tested in complementation crosses with different alleles of mus101.

| mus101 allele | Phenotype tested | p[w⁺, X9] | p[w⁺, B10] |
|---|---|---|---|
| mus101^{D1} | MMS sensitivity | + | − |
| mus101^{D2} | MMS sensitivity | + | − |
| mus101^{K451} | MMS sensitivity | + | − |
| mus101^{lcd} | lethality | − | − |
| mus101^{sm} | lethality | + | − |
| mus101^{tsl} | temperature sensitivity | + | − |

(+) indicates that the phenotype is rescued, (−) that it is not.

Genomic Sequence of Mus101

The complete sequence of mus101 was achieved by sequencing the distal 6 kb of the X9 genomic restriction fragment (FIG. 6). The X9 fragment was cloned in the vector; pBluescript, and both ends were sequenced using commercially available primers. The distal part of X9 overlaps with cDNA “A” (620 bp), and the proximal part (about 700 bp) overlaps with cDNA “C”, corresponding to garnet (sequences not shown, diagrammatic representation in FIG. 4B). To extend the sequence from the distal part of X9 to the mus101 complete genomic sequence, I used the primers previously synthesised for sequencing mus101 cDNAs as well as primers designed specifically for the X9 fragment (Materials and Methods). A total of 5977 nucleotides were sequenced, from the distal part of the X9 fragment, extending through mus101 coding sequence to meet up with the its 3′ non-coding region.

The mus101 genomic sequence predicts an ORF of 1425 amino acids, with a predicted molecular weight of 158 kDa. The predicted protein is composed of 169 strongly basic, 181 strongly acid, 442 hydrophobic and 396 polar amino acids. The calculated isoeletric point is 6.4 and the charge at pH 7 is −8.8. There are three in-frame methionines in the N terminus of the predicted protein, in positions 1, 5 and 7. By the analysis of the nucleotide sequence upstream of the ATGs coding for the methionines in the beginning of the predicted Mus101 protein, it is likely that the first ATG is the translation start site. Of 10 nucleotides, 5 match the consensus sequence (CACAACCAAAATG, Cavener and Ray, 1991), including the most conserved “CA” at positions −4 and −3, respectively, and three other nucleotides match the second best option. The region upstream of the ATG at positions 3 and 7 have three nucleotides identical to the consensus sequence, including the most conserved A at position −3 and four nucleotides match the second best option. The coding region is followed by 548 nucleotides of 3′ non-coding region, including four consensus polyadenylation signals (AATAAA—FIG. 6, bold).

The 5′ mus101 sequence is represented by 408 bp upstream of the first ATG in FIG. 6. It is likely to contain at least part of the promoter region since it has two DRE (Drosophila DNA replication-related element) motifs, localised at positions −377 to −370 and −352 to −345 with respect to the first ATG (FIG. 6, blue). DRE is a cis-acting positive regulatory element present in the promoters of a variety of genes, including those encoding proteins with functions in DNA replication, transcription, translation, signal transduction and cell cycle (Matsukage et al., 1995). It is represented by the palindromic sequence 5′-TATCGATA. Other genes containing DRE motifs include DNA polymerase a 180-kDa and 73-kDa subunits, PCNA and Cyclin A. It has been shown by both in vivo and in vitro experiments that DRE motifs are required for the activities of promoters of these genes. The activity is mediated by DREF, a factor that associates specifically with DRE sequences.

The presence of DRE sequences in the promoter of the mus101 gene strongly suggests that these sequences are important for its transcription, as is the case for other genes required for DNA replication.

The Drosophila PCNA gene promoter contains at least four transcriptional regulatory elements: besides DRE, the regulatory elements E2F, URE (Upstream Regulatory Element) and CFDD (Common regulatory Factor for DNA replication and DREF genes) have been demonstrated to be important. Specific protein factors bind to each one of these regulatory elements. Of these regulatory elements, only CFDD (other than DRE) is found in mus101 promoter region. The CFDD sequence present in mus101 promoter is 5′CGATA, and overlaps with the DRE elements. In the PCNA promoter, the CFDD sequence has been shown to promote transcription independently of DRE, even though they have overlapping recognition sequences.

Mus101 Predicted Protein has 7 BRCT Domains

The Mus101 predicted protein has seven regions of repeated BRCT domains. BRCT stands for BRCA1 C-Terminus. The motif was originally noted as a repeated region in the carboxy-terminus of the human ovarian and breast cancer gene BRCA1; in the budding yeast DNA repair and checkpoint protein Rad9; and in a human protein that binds to p53, named 53BP1 (Koonin et al., 1996). The use of more sophisticated computer programs was later able to identify BRCT domains in a larger number of proteins (Altschul et al., 1997; Bork et al., 1997; Callebaut and Mornon, 1997). Among the proteins containing BRCT domains, in addition to those previously reported by Koonin et al., 1996 are: XRCC1, Rad4, Ect2, Rev1, Crb2, Rap1, terminal deoxynucleotidyltransferases (TdT), human DNA ligases III and IV (Bork et al., 1997; Callebaut and Mornon, 1997), and TopBP1, (Yamane et al., 1997). The majority of these proteins are involved in DNA repair and cell cycle checkpoint control.

For example, Rad9 is a budding yeast protein required to mediate G1 and G2 checkpoint arrest following DNA damage. The human XRCC1 encodes a protein involved in rejoining of DNA single-strand breaks that arise following treatment with alkylating agents or ionising radiation. The fission yeast Rad4/Cut5 protein is necessary for arrest in response to incomplete DNA replication and Crb2 is a protein that interacts with Cut5 and is required for both DNA damage and replication checkpoints.

A BRCT domain is composed of approximately 100 amino acids with a characteristic hydrophobic profile which is easily identified in a HCA plot (Hydrophobic cluster analysis) (Callebaut and Mornon, 1997). The HCA of the Mus101 predicted protein showing the localisation of the BRCT domains is given in FIG. 7. In fact, the similarities between BRCT domains are more pronounced when the HCA of the protein is examined, rather than in its amino acid sequence. As an example (FIG. 8) similarities are shown between the highly conserved BRCT domain I in Mus101 and TopBP1, C elegans clone F37D6.1, Rad4 and Ect2, by both alignment of the HCA plots (A) and amino acid sequence (B).

The number and position of BRCT domains vary in different proteins. BRCA1, Rad9, XRCC1 and Crb2 have two domains in their C-termini, Ect2 has two domains in its N-terminus, and Rad⁴/Cut5 and Dpb11 have two domains in their N-termini and two domains in the central part of the protein (Bork et al., 1997; Callebaut and Mornon, 1997). The majority of the proteins described so far have on average two to four BRCT domains, although some have a greater number, like the C. elegans clone F37D6.1, of unknown function, which has 6 domains (Bork et al., 1997; Callebaut and Mornon, 1997). The human TopBP1 protein has eight BRCT domains, the greatest number of domains so far observed in a single protein (Yamane et al., 1997). Mus101 has seven BRCT domains, and its distribution is similar to the TopBP1, with only the central BRCT of TopBP1 missing in Mus101 (FIG. 9). The position of the Mus101 BRCT domains are: BRCT I: 121-205, BRCT II: 217-304, BRCT III: 397-485, BRCT IV: 605-697, BRCT V: 708-792, BRCT VI: 1206-1293 and BRCT VII: 1328-1409. The BRCT domains I, III, V and VI are represented in green and the BRCT domains II, IV and VI in red in FIG. 6. A comparison of the localisation of BRCT domains in Mus101, TopBP1, F37D6.1, rad4/Cut5 and Ect2 is presented in FIG. 8C.

It has been suggested by Callebaut and Mornon, 1997 and Bork et at., 1997 that the BRCT domains may be the sites for interaction between proteins implicated in the maintenance of the genome in response to damage. The knowledge of such interactions is beginning to bring light upon poorly understood repair mechanisms. Examples are accumulating, showing that BRCT domain containing proteins can participate in different aspects of DNA repair and cell cycle checkpoint control.

The protein encoded by the breast and ovarian cancer gene BRCA1 interacts in vivo with BARD1 (BRCT-Associated Ring Domain) protein (Wu et al., 1996). BARD1 and BRCA1 have similar structural organisation. Both proteins have ring fingers at their N-termini, and two BRCT domains at their C-termini (Koonin et al., 1996; Wu et al., 1996). The interaction between these proteins is through their N-terminal regions (Wu et al., 1996). The N-terminus of BRCA1 also interacts with the C-terminus of p53, and it is suggested that BRCA1 can enhance p53dependent gene expression acting as a p53 coactivator. Recently, BRCA1 was found to be a component of the RNA polymerase II holoenzyme by its association with RNA helicase A. The interaction between these proteins is made through the BRCA1 C-terminus, where two BRCT domains are present. The interaction between BRCA1 and RNA polymerase II and the requirement of BRCA1 for transcription-coupled repair of oxidative damage reinforces the model that BRCA1 acts as a transcriptional coactivator.

The N-terminal region of Rad4/Cut5, which has two repeated BRCT domains, interacts with the N-terminus of Crb2 (for Cut5-repeated binding), a protein required for checkpoint arrest induced by UV irradiation and DNA polymerase mutants. The interaction is dependent on the BRCT domain, since a construct containing the amino acid substitution T45M, in the first BRCT domain of Rad4/Cut5 (the same mutation observed in rad4⁺/cut5⁺ mutants) is not able to interact in a two-hybrid system with Crb2. Rad4/Cut5 and Crb2 also interact with Chk, and it has been suggested by these authors that the three proteins may form a checkpoint sensor-transmitter pathway to arrest the cell cycle.

The human proteins XRCC1 and DNA ligase III are also physically associated and interact in vitro and in vivo. The association between XRCC1 and DNA ligase III is made through the C-terminal region of both proteins, where their BRCT domains are located. There are two distinct forms of DNA ligase m, α and β, that are a result of different mRNA splicing. Only the DNA ligase III α, which contains a BRCT domain in its C-terminus associates with XRCC1. The human protein XRCC1 also interacts with DNA polymerase-β. A possible interaction between XRCC1 and poly (ADP-ribose) polymerase (PARP) has been suggested previously. XRCC1 interacts with PARP through its central region, which contains a BRCT domain. The importance of the BRCT domains in XRCC1 is further highlighted by the identification of mutations in four mutant cell lines of hamster XRCC1 that either alter or disrupt the BRCT domains.

The DNA repair protein XRCC4, is involved in repair of DNA double-strand breaks and in V(D)J recombination. XRCC4 is a nuclear phosphoprotein and is an effective substrate for DNA-PK in vitro. XRCC4 associates with DNA ligase IV, via the DNA ligase IV C-terminal BRCT domains. Results obtained previously suggest that XRCC4 acts as a molecular bridge to target DNA ligase IV to other components of the DNA non-homologous end-joining apparatus, and implicate DNA ligase IV in the joining of double-strand breaks via the non-homologous end-joining pathway and in the ligation steps of V(D)J recombination.

Recently a protein (TopBP1) was described that interacts in vitro with the C-terminus of DNA Topoisomerase II (Topo II) β (Yamane et al., 1997). The interaction between DNA Topo II β and TopBP1 (Topoisomerase-IIβ-Binding Protein 1) is made by the C-terminus of TopBP1, where two consecutive BRCT domains are located (Yamane et al., 1997): Although the DNA Topo II is very conserved among different organisms, its C-terminus is quite divergent, suggesting that these segments may, mediate different cellular functions. Since one of the functions of Topo II is to make transient breaks in DNA during its catalytic reaction, and due to the fact that TopBP1 has BRCT domains, Yamane et al., 1997 have suggested that TopBP1 may be involved in repair of DNA-strand breaks caused by failure of the catalytic reactions of Topo II.

Mus 101 Predicted Protein is Similar to the Juman Protein TopBP1

A database search using the PSI-blast program (Altschul et al., 1997) on Mus101 predicted protein reveals the highest scores for a human protein first described as having similarity to the fission yeast Rad4/Cut5 gene product (Nagase et al., 1996). This human protein is identical to TopBP1 (Yamane et al., 1997). In addition to TopBP1 and Rad4/Cut5, the proteins showing significant similarity to Mus101 predicted protein are a C elegans clone F37D6.1, an A. thaliana clone T10M13.12, the mouse transforming protein Ect2, and the human DNA repair protein XRCC1.

All these proteins have BRCT domains. In fact, the regions of highest similarity that these proteins show with Mus101 are in the BRCT domains, with the exception of TopBP1 and the C. Elegans clone F37D6.1, where the regions of similarity extend beyond the BRCT domains. FIG. 9 shows the pairwise combination of Mus101 and TopBP1 using the Blast 2 program (Altschul et al., 1997). Both proteins have similar, size: the Mus101 predicted protein is 1425 amino acids long, and the TopBP1 predicted protein is 1551 amino acids long. The N-terminal and central part of Mus101 has 26% identity and 44% similarity to the N-terminus of TopBP1. This region contains the Mus101 and TopBP1 BRCT domains I, II, III, IV and V. The C-terminus of Mus101 has 35% identity and 55% similarity with the C-terminus of TopBP1. This region contains the Mus101 BRCT domains VI and VII and the TopBP1 BRCT domains VII and VIII. Thus, the region of TopBP1 which contains the BRCT domain VI does not share homology to Mus101.

BRCT domains vary in number, position and sequence in different proteins. The BRCT domain I seems to be the most conserved domain among different proteins. The BRCT domain I of Mus101 is similar to the BRCT I domains of TopBP1 protein, C. elegans clone F37D6.1, mouse Ect2 and to fission yeast Rad4/Cut5 (FIG. 8).

The region between amino acids 817 and 1218 of Mus101 protein that does not have similarity to the human TopBP1 contains two distinct sub-regions with similarity to other proteins. The amino acid sequence 820-956 of Mus101 has 27% identity and 46% similarity to the C-terminus of human treacle protein (TCOF1), a putative nucleolar trafficking phosphoprotein which is defective in patients with Treacher Collins syndrome, a craniofacial developmental disease (Wise et al., 1997). The amino acid sequence 949 to 1130 of the Mus101 predicted protein is 26% identical (38% similar) to the C-terminus of Drosophila posterior sex combs (Psc) protein. Psc is a product of one of the Polycomb group of genes (Pc-G). Pc-G genes are needed to maintain expression patterns during Drosophila development, by the repression of target genes (for review see Pirrota, 1995).

Discussion

mus101 mutations show a wide range of phenotypes. There are three lethal, one female sterile and two mutagen sensitive alleles. Analysis of brains of the late lethal alleles mus101^{tsl} and mus101^{lcd} show an abnormal undercondensation of heterochromatic regions (Axton, 1990; Gatti et al., 1983). The mutagen sensitive allele mus101^{D1} is defective in post-replication repair (Boyd and Setlow, 1976). The female sterile allele mus101^{K451} is defective in chorion protein gene amplification (Komitopoulou et al., 1983; Komitopoulou et al., 1988; Orr et al., 1984), a form of differential replication of gene clusters in follicle cells which occurs during oogenesis (Spradling, 1981; Spradling and Mahowald, 1980). The analysis of these phenotypes suggests that the wild type Mus101 protein might be involved in different aspects of chromatin structure, DNA replication and repair. However it may be difficult to distinguish cause and consequence. Is failure in DNA replication responsible for the undercondensed chromatin observed in the alleles mus101^{tsl} and mus101^{lcd}, or is the failure of proper condensation responsible for DNA replication problems? We approached the cloning of mus101 to try to elucidate these questions, hoping that this would permit a molecular analysis of the gene product.

We have now presented data regarding the localisation of the mus101 gene to a 10 kb BamHI fragment (B10) in the proximal part of cloned DNA fragments from a chromosome walk (FIG. 5). This enabled us to isolate partial cDNAs corresponding to the mus101 transcription unit, to prove by germ line transformation that this was the mus101 gene and to determine its genomic sequence (FIG. 6).

The genomic sequence of mus101 is 5276 bp long and predicts a single ORF of 1425 amino acids. I cannot be certain of the precise position of the N-terminus of the protein since there are three methionines at positions +1, +3 and +7. It is possible that the ATG corresponding to the methionine at position +1 is the one used to initiate translation, since the region upstream of it has more similarities to the consensus sequence for translation initiation in Drosophila. It is also possible, that the ATG corresponding to the methionines at positions +3 and +7 are used to initiate translation, maybe in a less effective way, since the region upstream of these ATGs has considerable similarity to the Cavener consensus sequence. I have assigned the Met at position +1 as a potential N-terminal residue. To definitively identify the initiation methionine it would be necessary to perform N-terminal sequencing of the purified Mus101 protein. A definitive indication of gene organisation will necessitate the recovery of a full length cDNA or performing primer extension experiments. The low number of cDNAs recovered in the screening of embryonic libraries, suggests that mus101 or is expressed at low levels during this stage of development. However, mus101 is expressed at high levels in the ovarian tissue, since I have recently recovered dozens of mus101 clones from an ovarian cDNA library (data not shown). The characterisation of these clones is under way.

Two DRE sequences in the region 350-380 nucleotides upstream of the first methionine (FIG. 6, blue) suggest that this region might be part of the promoter. DRF sequences are found in the promoter of genes essential for DNA replication, such as PCNA and DNA polymerase cc (subunits 73 kDa and 180 kDa). It is not surprising to find replication related sequences in the mus101 promoter, since mus101 is implicated in DNA replication from the analysis of the mutant phenotype of the female-sterile allele mus101^{K451}. Promoter activity assays need to be undertaken to determine if these DRE sequences are functional.

Multiple promoter elements are necessary for the efficient transcription of the Drosophila PCNA gene: the DRE, URE, E2F and CFDD elements and their specific binding factors, DREF, UREF, E2F/DP and CFDD, respectively. Recognition sequences for E2F, DRE and CFDD were also identified in the promoters of the genes encoding the Drosophila DNA polymerase α 180 kDa and 73 kDa subunits. By comparison, the potential promoter region of mus101 that I have sequenced has two sequences for CFDD motifs, which overlap the DRE sequences. The observed differences between the promoter region of mus101 and the promoter regions of PCNA and DNA polymerase α 180 kDa and 73 kDa subunits genes could mean either that insufficient upstream DNA of mus101 has been sequenced or that mus101 needs alternative factors to activate its transcription. To fully understand the transcriptional activation of genes involved in DNA replication, the promoters from a larger number of genes involved in DNA replication should be analysed.

The presence of seven BRCT domains in the predicted Mus101 coding region (FIGS. 6 and 7) places the gene into the BRCT superfamily, associated with DNA repair and maintenance of the genome (Bork et al., 1997; Callebaut and Mornon, 1997).

The Mus101 predicted protein has highest scores of similarity to TopBP1, a human protein that binds to Topo II β (Yamane et al., 1997) and which has eight BRCT domains (FIG. 9). Mus101 predicted protein is also significantly similar to the fission yeast protein Rad4/Cut5, to the human XRCC1, the mouse oncoprotein Ect2 and to the C. elegans clone F37D6.1. The regions with similarity between Mus101 predicted protein and Rad4/Cut5, XRCC1 and Ect2 are restricted to the BRCT domains. The Mus101 predicted protein has regions of similarity that extend beyond the BRCT domains in the human TopBP1 and the C elegans clone F37D6.1. The similarities between Mus101 predicted protein and TopBP1 lie in the N-terminal and central regions and in the C-terminal region (FIG. 9). One region of Mus101 which is not similar to TopBP1 does share some similarity to the human TCOF1 protein and to the Drosophila Psc protein. However, as no specific functional domains have been identified within these regions, the significance of this is still to be determined.

Since the similarity between the predicted Mus101 protein and TopBP1 is so extensive, it is possible that Mus101 is a functional homologue of TopBP1. The C-terminal region of TopBP1 that interacts with Topo II β has 35% identity and 55% similarity with the predicted Mus101 protein (FIG. 8B).

DNA topoisomerases II catalyse topological changes in DNA to enable decatenation to facilitate the completion of DNA replication, and chromatid separation. Failure of these processes would lead to defects in chromatin organisation. In Drosophila, DNA Topo II is present in at least three separate functional pools: one for chromosome condensation, one for chromosome segregation and one pool which remains associated with the chromosome throughout the cell cycle. Recently, the Drosophila Topo II was identified as a component of the chromatin-remodelling factor CHRAC (CHRomatin-Accessibility Complex, Vargas-Weiz et al., 1997), suggesting a structural role for this protein in chromatin organisation. DNA Topo II is associated in Drosophila with Barren, a protein necessary for chromatin segregation at anaphase (Bhat et al., 1996).

The structure of DNA topoisomerases is conserved through evolution. The N-terminal region is the ATPase functional domain, and the central region is the DNA breakage and reunion activity domain. The C-terminus of DNA topoisomerases is the least conserved region, and may specify differences in different isoenzymes. Although the C-terminal regions of Topo II are not conserved at the amino acid level, this region is extremely hydrophilic and charged among eukaryotic enzymes (Wyckoff et al., 1989). It is the C-terminal region of the human Topo II β that interacts with TopBP1.

It is possible that Mus101 is a functional homolog of TopBP1, and that Mus101 also binds to DNA Topo II. It might be also possible that the putative interaction between Mus101 and Topo II is specific to heterochromatic regions. Once this interaction is disrupted, the heterochromatic regions fail to condense properly, the phenotype observed in mus101 mutants. If true, this could explain the altered condensation of heterochromatic regions observed in mitotic cells from ganglia in the lethal lines mus101^{lcd} and mus101^{ts}.

To address this question, antibodies that identify the Mus101 protein could be used to determine the subcellular localisation of the Mus101 protein, and determine if it matches with the distribution of Topo II, and to determine the distribution of the protein in the mutants mus101^{lcd} and mus101^{ts}. Such antibodies would also enable immunoprecipitation experiments to investigate direct interaction between Topo II and Mus101 protein. If physical interaction can be established between the proteins, than it will be possible to determine whether this can modulate Topo II activity.

The Mus101 BRCT domains I and II are very similar to the BRCT domains I and II of the oncogene product Ect2 and the fission yeast replication checkpoint protein Rad4/Cut5 (FIG. 8), regions that are important to the roles of these proteins in cell cycle control. Truncation of the N-terminal region of Ect2, to remove these two BRCT domains, increases its transforming activity, suggesting that this region has a negative effect on cell division. The N-terminal domain of Rad4/Cut5 containing these BRCT domains is essential for complementation of the temperature sensitive phenotype in cut5 mutants. Moreover, its overexpression blocks cell division (Saka and Yanagida, 1993). The localisation of the mutations in independent rad4⁺/cut5⁺ alleles reveals a consistent amino acid substitution at the 45th codon, in the conserved stretch VTHLIA in BRCT domain I. This amino acid substitution prevents the interaction of the Rad4/Cut5 protein with the DNA replication and DNA damage checkpoint protein Crb2.

mus101 and rad4⁺/cut5⁺ mutants have similar phenotypes. Both are mutagen sensitive and are defective in DNA repair. They also both encode members of the BRCT superfamily, with similar BRCT domains I and II. The Rad4/Cut5 gene product is a component of the replication checkpoint control system) and together with Crb2 forms a complex with the checkpoint protein Chk1. It has been suggested that these proteins may form a checkpoint sensor-transmitter pathway to arrest cell cycle.

mus101 may be necessary for the DNA replication checkpoint. This could be assessed by in vivo and in vitro experiments to assess the response of mus101 alleles to drugs that block DNA replication. In S. pombe, the replication checkpoint is categorised into two classes: one resulting from a defect in the replication machinery (mutations in polymerases and ligases), and the other caused by the limitation of normal nucleotide supply (HU and cdc22 mutation). Rad4/Cut5 is necessary for both checkpoints, whereas Crb2 is necessary only for the nucleotide supply checkpoint. It would be interesting to investigate if mus101 is necessary for any of these replication checkpoints. The mutant mus101^{D1} has been tested for sensitivity to HU (Banga et al., 1986). This study revealed that the DNA replication checkpoint appears to be still intact in this mutant. It is important to check the HU sensitivity of other mus101 alleles, such as mus101^{tsl} and the female sterile allele mus101^{k451} and to determine whether the checkpoint caused by a defect in the replication machinery is present.

A number of proteins belonging to the BRCT superfamily are involved in different aspects of DNA repair. For example, the human protein XRCC1 is necessary for repair of single strand breaks and the protein XRCC4 is necessary for double strand break repair and for V(D)J recombination The study of the interaction of these proteins with other proteins, that may or not contain BRCT domains, offers possibilities to elucidate these repair mechanisms.

XRCC1 interacts with DNA ligase m, PARP and DNA polymerase β 1996. Although the mechanism of action of these proteins is not understood, it seems reasonable that the four proteins may form a complex that acts in base excision repair. XRCC4 interacts with DNA ligase IV, and it has been suggested that the function of the XRCC4DNA ligase IV complex may be to carry out the final steps of V(D)J recombination and joining of DNA ends.

The mutagen sensitive allele mus101^{D1} is partially defective in postreplication repair (Boyd and Setlow, 1976). The mechanism of postreplication repair is not well understood in higher eukaryotes. It may be possible to try to elucidate the process of postreplication repair by identifying proteins that interact with mus101, determining the sites of such interactions, and by analysing the nature of the mus101^{D1} mutation.

References

Altschul, S. F., Madden, T. L., Schaffer, A., Zhang, J., Zhang, Z., Miller, W., and Lipman, D. J. (1997). Gapped BLAST and PSI-BLAST: a new generation of protein database search programs. Nucleic Acids Res., 25: 3389-3402.

Ashburner, M. (1989). Drosophila, a laboratory manual (Cold Spring Harbour: Laboratory Press).

Axton, M. (1990). Genetic and molecular analysis of mitotic chromosome condensation in Drosophila. Ph.D. Thesis (London, Imperial College of Science and Technology).

Baker, B. S., and Smith, D. A. (1979). The effects of mutagen-sensitive mutants of Drosophila melanogaster in nonmutagenized cells. Genetics, 92: 833-847.

Banga, S. S., Shenkar, R., and Boyd, J. B. (1986). Hypersensitivity of Drosophila mei-41 mutants to hydroxyurea is associated with reduced mitotic chromosome stability. Mutation Res., 163: 157-165.

Bhat, M. A., Philp, A. V., Glover, D. M., and Bellen, H. J. (1996). Chromatid segregation at anaphase requires the barren product, a novel chromosome-associated protein that interacts with Topoisomerase II. Cell, 87: 1103-1114.

Bork, P., Hofmann, K., Bucher, P., Neuwald, A. F., Altschul, S. F., and Kooning, E. V. (1997). A superfamily of conserved domains in DNA damage-responsive cell cycle checkpoint proteins. FASEB J, 11: 68-76.

Boyd, J. B., Golino, M. D., Nguyen, T. D., and Green, M. M. (1976). Isolation and characterization of X-linked mutants of Drosophila melanogaster which are sensitive to mutagens. Genetics, 84: 485-506.

Boyd; J. B., Golino, M. D., and Setlow, R. B. (1976). The mei-9a mutant of Drosophila melanogaster increases mutagen sensitivity and decreases excision repair. Genetics, 84: 527-544.

Boyd, J. B., Golino, M. D., Shaw, C. V., Osgood, C. J., and Green, M. M. (1981). Third-choromosome mutagen-sensitive mutants of Drosophila melanogaster. Genetics, 97: 607-623.

Boyd, J. B., Mason, J. M., Yamamoto, A. H., Brodberg, R. K., Banga, S. S., and Sakaguchi, K. (1987). A genetic and molecular analysis of DNA repair in Drosophila. J. Cell Sci. Supplement, 6: 39-60.

Boyd, J. B., and Setlow, R. B. (1976). Characterization of postreplication repair in mutagen-sensitive strains of Drosophila melanogaster. Genetics, 84: 507-526.

Brown, T. C., and Boyd, J. B. (1981). Postreplication repair-defective mutants of Drosophila melanogaster fall into two classes. Mol. Gen. Genet., 183: 356-362. Callebaut, I., and Mornon, J.-P. (1997). From BRCA1 to RAP1: a widespread BRCT module closely associated with DNA repair. FEBS Letters, 400: 25-30.

Cavener, D. R., and Ray, S. C. (1991). Eukaryotic start and stop translation sites. Nucleic Acids Res., 25: 3185-3192.

Fenech, M., Carr, A. M., Murray, J., Watts, F. Z., and Lehmann, A. R. (1991). Cloning and characterization of the rad4 gene of Schizosaccharomyces pombe, a gene showing short regions of sequence similarity to the human XRCC1 gene. Nucleic Acids Res., 19: 6737-6741.

Gatti, M. (1979). Genetic control of chromosome breakage and rejoining in Drosophila melanogaster: spontaneous chromosome aberration in x4inked mutants defective in DNA metabolism. Proc. Natl. Acad Sci. (USA), 76: 1377-1381.

Gatti, M., Smith, D. A., and Baker, B. S. (1983). A gene controlling condensation of heterochromatin in Drosophila melanogaster. Science, 221: 83-85.

Gowen, L. C., Avrustskaya, A. V., Latour, A. M., Koller, B. H., and Leadon, S. A. (1998). BRCA1 required for transcription-coupled repair of oxidative damage. Science, 281: 1009-1012.

Greenwell, P. W., Kronmal, S. L., Porter, S. E., Gassenhuber, J., Obermaier, B., and Petes, T. D. (1995). TEL1, a gene involved in controlling telomere length in S. cerevisiae, is homologous to the human Ataxia Telangiectasia gene. Cell, 82: 823-829.

Henderson, D. H., Bailey, D. A., Sinclair, D. A. R., and Grigliatti, T. A. (1987). Isolation and characterization of second chromosome mutagen-sensitive mutations in Drosophila melanogaster. Mutation Res., 177: 83-93.

Karess, R. E. (1985). P-element mediated germline transformation of Drosophila. In DNA cloning, D. M. Glover, ed. (Oxford: IRL Press).

Komitopoulou, K., Gans, M., Margaritis, L. H., Kafatos, F. C., and Masson, M. (1983). Isolation and characterization of sex-linked female-sterile mutants in Drosophila melanogaster with special attention to eggshell mutants. Genetics, 105: 897-920.

Komitopoulou, K., Margaritis, L. H., and Kafatos, F. C. (1988). Structural and biochemical studies on four sex-linked chorion mutants of Drosophila melanogaster. Dev. Genet., 9: 37-48.

Koonin, E. V., Altschul, S. F., and Bork, P. (1996). BRCA1 protein products: functional motifs. Nature Genet., 13: 266-268.

Levis, R., Hazelrigg, T., and Rubin, G. M. (1985). Separable cis-acting elements for expression of the white gene of Drosophila. EMBO J, 4: 3489-3499.

Lindsley, D., and Zimm, G. (1992). The genome of Drosophila melanogaster (New York: Academic Press).

Matsukage, A., Hirose, F., Hayashi, Y., Hamada, K, and Yamaguchi, M. (1995). The DRE sequence TATCGATA, a putative promoter-activating element for Drosophila melanogaster cell-proliferation-related genes. Gene, 166: 233-236.

Nagase, T., Ishikawa, K., Miyajima, N., Tanaka, A., Kotani, H., Nomura, N., and Ohara, O. (1998). Prediction of the coding sequences of unidentified human genes. IX. The complete sequences of 100 new cDNA clones from brain which can code for large proteins in vitro. DNA Res., 5: 31-39.

Nagase, T., Seki, N., Ishikawa, K., Ohira, M., Kawarabayasi, Y., Ohara, O., Tanaka, A., Kotani, H., Miyajima, N., and Nomura, N. (1996). Prediction of the coding sequences of unidentified human genes. VI. The coding sequences of 80 new genes (KIAA0201-KIAA0280) deduced by analysis of cDNA clones from cell line KG-1 and brain. DNA Res., 3: 321-329.

Orr, W., Komitopoulou, K., and Kafatos, F. (1984). Mutants supressing in trans chorion gene amplification in Drosophila. Proc. Natl. Acad. Sci (USA), 81:. 3773-3777.

Orr-Weaver, T. L. (1991). Drosophila chorion genes: cracking the eggshell's secrets. BioEssays, 13: 97-105.

Perrimon, N., and Gans, M. (1983). Clonal analysis of the tissue specificity of recessive female-sterile mutations of Drosophila melanogaster using a dominant female-sterile mutation fs(1)K1237. Developmental Biology, 100: 365-373.

Pirrota, V. (1995). Chromatin complexes regulating gene expression in Drosophila. Current Opinion in Genetics & Development, 5: 466-472.

Pirrota, V. (1988). Vectors for P-element transformation. In Drosophila: A pratical approach, R. L. Rodriguez and D. T. Denhardt, eds. (Boston and London: Butterworths), pp. 437-456.

Poole, S. J., Kauvar, L. M., Drees, B., and Kornberg, T. (1985). The engrailed locus of Drosophila: structural analysis of an embryonic transcript. Cell, 40: 37-43.

Roberts, D. B. (1986). Drosophila: a pratical approach (Oxford: IRL Press).

Saka, Y., and Yanagida, M. (1993). Fission yeast cut5+, required for S phase onset and M phase restraint, is identical to the radiation-damage repair gene rad4+. Cell, 74: 383-393.

Sambrook, J., Fritsch, E. F., and Maniatis, T. (1989). Molecular cloning: a Laboratory manual (Cold Spring Harbour: Laboratory Press).

Smith, D. (1976). Mutagen sensitivity of Drosophila melanogaster. Mol. Gen. Genet., 149: 73-85.

Smith, D. A., Baker, B. S., and Gatti, M. (1985). Mutations in genes encoding essential mitotic functions in Drosophila melanogaster. Genetics, 110: 647-670.

Snyder, R D., and Smith, P. D. (1982). Mutagen sensitivity of Drosophila melanogaster. V. Identification of second chromosomal mutagen sensitive strains. Mol. Gen. Genet., 188: 249-255.

Spradling, A. C. (1981). The organization and amplification of two chromosomal domains containing Drosophila chorion genes. Cell, 27: 193-201.

Spradling, A. C., and Mahowald, A. P. (1980). Amplification of genes for chorion proteins during oogenesis in Drosophila melanogaster. Proc. Natl. Acad Sci. (USA), 77: 1096-1100.

Vargas-Weiz, P. D., Wilm, M., Bonte, E., Dumas, K., Mann, M., and Becker, P. B. (1997). Chromatin-remodelling factor CHRAC contains the ATPases ISWI and Topoisomerase II. Nature, 388: 598-602.

Wise, C. A., Chiang, L. C., Pazaekas, W. A., Sharma, M., Musy, M. M., Ashley, J. A., Lovett, M., and Jabs, E. W. (1997). TCOF1 gene encodes a putative nuclear phosphoprotein that exhibits mutations in Treacher Collins Syndrome throughout its coding region. Proc. Natl. Acad Sci. (USA), 94: 3110-3115.

Wu, L. C., Wang, Z. W., Tsan, J. T., Spillman, M. A., Phung, A., Xu, X. L., Yang, M. C., Hwang, L. Y., Bowcock, A. M., and Baer, R. (1996). Identification of a ring protein that can interact in vivo with the BRCA1 gene product. Nature Genet., 14: 430440.

Wyckoff, E., Natalie, D., Nolan, J., Lee, M., and Hsieh, T.-s. (1989). Structure of the Drosophila DNA Topoisomerase II gene. Nucleotide sequence and homology among Topoisomerases II. J. Mol. Biol., 205: 1-13.

Yamamoto, Y., Girard, F., Bello, B., Affolter, A., and Gehring, W. J. (1997). The cramped gene of Drosophila is a member of the Polycomb-group, and interacts with mus2O9, the gene encoding Proliferating Cell Nuclear Antigen. Development, 124: 3385-3394.

Yamane, K., Kawabata, M., and Tsuruo, T. (1997). A DNA-topoisomerase-II-binding protein with eight repeating regions similar to DNA-repair enzymes and to cell-cycle regulator. Eur. J Biochem., 250: 794-799.

1. A polynucleotide encoding mus101 or a homologue thereof.

2. A polynucleotide selected from:
(a) polynucleotides comprising the nucleotide sequence set out in SEQ ID No. 1 or the complement thereof.
(b) polynucleotides comprising a nucleotide sequence capable of hybridising to the nucleotide sequence set out in SEQ ID No. 1, or a fragment thereof.
(c) polynucleotides comprising a nucleotide sequence capable of hybridising to the complement of the nucleotide sequence set out in SEQ ID No. 1 or a fragment thereof.
(d) polynucleotides comprising a polynucleotide sequence which is degenerate as a result of the genetic code to the polynucleotides defined in (a), (b) or (c).

3. A polynucleotide probe which comprises a fragment of at least 15 nucleotides of a polynucleotide as defined in claim 1 or 2.

4. A polypeptide which comprises the sequence set out in SEQ ID No 2, or a homologue, variant, derivative or fragment thereof.

5. A polynucleotide encoding a polypeptide according to claim 4.

6. A vector comprising a polynucleotide as defined in any one of claims 1, 2 or 5.

7. An expression vector comprising a polynucleotide as defined in any one of claims 1, 2 or 5, operably linked to a regulatory sequence capable of directing expression of said polynucleotide in a host cell.

8. An antibody capable of binding the polypeptide of SEQ ID. No. 2 or a fragment thereof.

9. A method for detecting the presence or absence of a polynucleotide as defined in any one of claims 1, 2 or 5 in a biological sample which comprises:
(a) bringing the biological sample containing DNA or RNA into contact with a probe according to claim 3 under hybridising conditions; and
(b) detecting any duplex formed between the probe and nucleic acid in the sample.

10. A method for detecting a polypeptide as defined in claim 4 present in a biological sample which comprises:
(a) providing an antibody according to claim 8;
(b) incubating a biological sample with said antibody under conditions which allow for the formation of an antibody-antigen complex; and
(c) determining whether antibody-antigen complex comprising said antibody is formed.

11. A polynucleotide according to any one of claims 1, 2 or 5 for use in therapy.

12. A polypeptide according to claim 4 for use in therapy.

13. An antibody according to claim 9 for use in therapy.

14. Use of a mus101 polypeptide or homologue, derivative, variant or fragment thereof in a method of identifying a substance capable of affecting mus101 function.

15. Use of a mus101 polypeptide or homologue thereof, or fragment in an assay for identifying a substance capable of increasing the susceptibility of a cell to DNA damaging agents.

16. A method for identifying a substance capable of binding to a mus101 polypeptide or a homologue, derivative, variant or fragment thereof, which method comprises incubating the mus101 polypeptide or homologue, derivative, variant or fragment thereof with a candidate substance under suitable conditions and determining whether the substance binds to the mus101 polypeptide or homologue, derivative, variant or fragment thereof.

17. A substance identified by the method of claim 14, 15 or 16.

18. A substance according to claim 17 for use in a method of inhibiting mitosis.

19. A substance according to claim 17 for use in a method of increasing the susceptibility of a cell to DNA damaging agents.

20. A process comprising the steps of:
(a) performing the method according to claim 14, 15 or 16; and
(b) preparing a quantity of those one or more substances identified as being capable of binding to a mus101 polypeptide or homologue, derivative, variant or fragment thereof.

21. A process comprising the steps of:
(a) performing the method according claim 14, 15 or 16; and
(b) preparing a pharmaceutical composition comprising one or more substances identified as being capable of binding to a mus101 polypeptide or homologue, derivative, variant or fragment thereof.

22. A method of treating a tumour which method comprises administering to a patient in need of treatment an effective amount of a substance capable of affecting mus101 function.

23. A method of increasing the susceptibility of a tumour cell to a DNA damaging agent which method comprises administering to said cell a substance capable of affecting mus101 function.


SEQUENCE LISTING (48 sequences)

SEQ ID NO: 1 - DNA, 5276 bp, Drosophila sp.
  CDS (409)..(4686)
ttgcctttcctttcttctttcttcctttcttttaggactgagctgccgtcgacgacgtggatatcgatatttcgcttaatgaaatatatcgatagcttaaaccaaaactgataaataactgcttattttcgaaatattttatttatttgataaaaagtaaataaaaaatccttacatcttagagataaataattaaaattacccatacacattattttttaattcgccgtcggcgttgccacatagctcaactcgtagttgccaactaatcacaagccagcgttgccaacggtttgaaggcaataacaaacggcgccaaaatagcgtacgcgaaaaaaagtgcagtgcgaaaatcacggcaattttgcagcgcatcacggagcagagaacacactcgcaaccgccatcatgagcataagcatgagcatggacgagaccatctgcgcgtacttcgtgaacaatctgaagccgggcgacggtggcgtccaggaggccgacacactgcagcaattcgaggcggcgcgggagctactgggccaacagttggcggaaacgcagatccggcaaataaagccaagcgaaggatatcccctgatcgcagccggtaaccttaccaagaaggacgtctttgtgctgacccagttcgagggcgaattcttcgagcaattgcagcagacgcgagcactaattctggggccaccgtgcctcatcacctgcctgcggcgcaatgaacccattcccgagggcagcagtgccatctacagcacggccatgcgggatctgcaggtctcggccacgggcataacaccacagaagaaagaggaattgagcaggctcataaactggatgggcggcatatactttcaaagcttcgggcatcgcaccacccacctcatttcgaacaccatcaagtccagcaagtacgagcaggcaacgctgaacggagtacccgtaatgcacgtcgactgggtgcagtacgtctgggatcagagtcgtcgcagccagcgcgagggcatcatggccacggatcctgatttcgataagtatcgcctgcccattttctttggtgcgaatatcacgtgcagtggattggatgtggcgcgcaaggatcaagttatgcggctggtcaacgataatggaggcatctatcatcgtgcctttcgctcccaggtggtggacatcgtcatcaccgagcaaacaaaaacggacaccgagaagtataaggcagccatacgctacaagaaggatgtcttgctgccggaatggatcttcgatagctgcaatcgcggctacgctctgcccacaaaggactatgaggtgcggcctggcaagacgtcgtccacacccaccaagaccacgcgtccgggcgcagctcccggtgcagatcaaacgcacctctcggatctctcacgtatcagcttcgtctccggctcgcgtcgcatgtgcagcgatcttagtaccgtcaacgaatccgtcagcagtgtgggcagcagttcgcccgccaagcagctgctcaagcaggcgactagcagtggccgcaactaccagcaggtgctggccgagattgaaccgcgtcaggcgaaaaaagcgggcgcctttctggatggctgctgtgtgtatttgagtggcttccgctcagaggagcgtgagaagctaaacagagtgctgaatacgggcggagcgacccgctacgatgaggccaatgagggcatctcacacatcattgtgggccaactggatgacgccgaataccgacagtggcagcgcgatggtctcatgggttcagttcatgtggtgcgcctagattggctgctggagagcattcgagctggtcgcgtggtcagtgagttggtgcatcgtgtgtcgatgccacagaatcgagaaccagacgttgcctctcctgccagcaagcgaacactgcgctccatgaaccacagcttcaagcagccaacattgcccatcaagaagaagcttttcgatcaggaaccggatcccgtgcaggaacaggagcacgaggagccggatcatacgctgctggatcagtactcacaggatcaaggagcagtggcacaactgccaccggcagatgttagtctccttcaaccagcggcaagttccacccaaatggatatacgccagcgagtctcggtagccaatccaaaaccacccgctgagggtttgcaattgccggatctcagtgccagcactctatccattgatttcgataagctggactacttcgccggtgtctctgtttatgtgcacagggagtgtttcaacgaggagttcttcaaccaaatgctaaccgaatgcgaagctgcccaaggcttattggtgccatcgagtttctccgatgaagtggatttcgccattgtcagctttgaggtagccttcgatgtgaagcaattacccgtcaaggcccgccatgtggtcaccgaactgttcctggaaagctgtatgaaaaagaatcaactgctgcccatcgaatattaccacaaacatgtgccggctaccgcactgcgtcagccgcttaagggaatgactattgtcgtatccatttatgcaggattggagcgggactttattaatgcgacagcagaactacttggcgcctccgtcaataagacattcatcaagaaggagaaaccgctgctggtgtgtcccagtgccgagggctccaagtatgaaggtgccatcaaatggaactatcccgtagtcacatccgattggctggtgcagtgcgcccgcactggtcagaagctgcccttcgttggatatttggtgggcaagagtcccgaggatttccccatatcgccacgcttgcgggacagcaatagccggacagcaagaagaccgaatgaatccacattggtggctcaaccggatgtaaccatggaggaggccgagaaccaaccggcgggatctgtcacaccagttactgctggcagtccaggagctcctgaactgacgcccctgcgcaacaagagagtttccgagctagctggaataccaggaggcagtgctcgtcatcgtggcaccagctccacatcttctccggactcaccatgcacgccacttagccaggtgggtgcccagcaatacaatctggacttcctagagcaattcgttcaacgcctggatacagaagagggcaaggattgtgtgcgcgagattatccgtgaaatgcgcgagaatcaaacgccggaattggaacgcattcgacggcaggcctgcacgcccgtcagtcgtaagcatcaacgaccagcaccgggaattccagatttttgtctcactcccgagttccagcagcggatggccgatgattttgagcggcgctggcgcctacccaccatgaaaatcaaaccagacacaccgttggccgtcatcaggcagcgcgtgatgcggatcacatgcgaaactctgggcatcgaatatgaagaaagtaatgctaagacgccaacgctatcggaatcgccatcaacggtaaagaagaagccgccaaccaggacgacgcaggccaccaaactcaactttgatagatcaccgaaaacaccgaagctatcgctaggtaaaaagacgccactccgggtgtccatgggttcaccccgcagcggaacgcaatcacccttcgtaccgaacacacagagtccaatcgaagcagcgccgccacggcgttcagatggtccaacgttaagcgaggaaggtcagagcactattaactttgacaagattagcttcgaggagtcggccgtgcccgtattggccccagatgttcccacagtggcgccagatgtcaagcaaatcaccgactatctaaagaactgcgaatcgcgaaggaacagtctgaagcgtagccacgacaacgacatggattgcggcgagagcgaggtgcagtatgtgcagcccttcgagtccgagggcttcgccctgggcaccgaggacatggtcgactggcgcgatccggcggagttcaatgcggcaaagagaagatcctctggcggttcgcccaagatgcagtatgctggcataccgtgcttcagcatctcgtgcggtgatgatgacgaaaagcgggcggaactaatcgcgcggatcacccaactgggcggaaaagtgtgcgagaatctggtgaactatgacgatagctgcacccatctgctgtgcgagcgtccgaatcgcggcgagaagatgctggcctgcattgcggccgggaaatggatactaaatatccagtatatcgaacagtcgcatgcgcgtggcgactttctggacgaaacgctgtacgaatggggcaacccgaaggccattaacttgcccaccctggcaccggaggaggagcccatcgctgccgccgtccaccgttggcgcacggagttgagtgcgtgcggcggtggcgccttctccgatcaccgggttatactcagcatgaacgagaggagcggggcgcccatcaggaatgtgctgcgtgccggcggcgcttgcatcctggagccaacaactccgttctccaaagatcctgtggccaaaagtgccagccattgttttgtcgatgtgaagaaggcgccgctgtcgacccaggacatggagtatcttcacaaatgtggtgtgcaggtgctcagccagattgccatcaacgcctatttgatgaacggcagggatgccgatctgggaaagtaccagctcctttgaacgaatcattagattctttgtacatgcattggattttttttttgttcctgttctatattttgatattttgtggttatataaaagttattgaattaaagccttcaaaggtttaatttaaaataggtctagtttaatcagttgtaagtgtaattttgaatttttgatttgtgtccttttcttttggttactgaaaaaaatcataatatttggaagcctttagcgtaatgaattactaataaattaatatcgttaattaacttcaattaacttcaaaataaacgaactgaaattgaagtccaaaaccatatgataattgtatttagtgatttatttctaaaggtgttgttgaaaattaattctaagtcaaagcgcaaattatttttgaaagaaatttgacgattttctatttttttattgaagctaccttagtttgtttcttttattaatttcattccaacttcaatttaataaacaaaatgtaatattaaaaacctaaagaaattctatttttttcatcccgatttcgatcaattacattatttattttccgcagccaaattaaacaataaatcaatatgcaagtgtattgt

SEQ ID NO: 2 - protein, 1425 aa, Drosophila sp.
MSISMSMDETICAYFVNNLKPGDGGVQEADTLQQFEAARELLGQQLAETQIRQIKPSEGYPLIAAGNLTKKDVFVLTQFEGEFFEQLQQTRALILGPPCLITCLRRNEPIPEGSSAIYSTAMRDLQVSATGITPQKKEELSRLINWMGGIYFQSFGHRTTHLISNTIKSSKYEQATLNGVPVMHVDWVQYVWDQSRRSQREGIMATDPDFDKYRLPIFFGANITCSGLDVARKDQVMRLVNDNGGIYHRAFRSQVVDIVITEQTKTDTEKYKAAIRYKKDVLLPEWIFDSCNRGYALPTKDYEVRPGKTSSTPTKTTRPGAAPGADQTHLSDLSRISFVSGSRRMCSDLSTVNESVSSVGSSSPAKQLLKQATSSGRNYQQVLAEIEPRQAKKAGAFLDGCCVYLSGFRSEEREKLNRVLNTGGATRYDEANEGISHIIVGQLDDAEYRQWQRDGLMGSVHVVRLDWLLESIRAGRVVSELVHRVSMPQNREPDVASPASKRTLRSMNHSFKQPTLPIKKKLFDQEPDPVQEQEHEEPDHTLLDQYSQDQGAVAQLPPADVSLLQPAASSTQMDIRQRVSVANPKPPAEGLQLPDLSASTLSIDFDKLDYFAGVSVYVHRECFNEEFFNQMLTECEAAQGLLVPSSFSDEVDFAIVSFEVAFDVKQLPVKARHVVTELFLESCMKKNQLLPIEYYHKHVPATALRQPLKGMTIVVSIYAGLERDFINATAELLGASVNKTFIKKEKPLLVCPSAEGSKYEGAIKWNYPVVTSDWLVQCARTGQKLPFVGYLVGKSPEDFPISPRLRDSNSRTARRPNESTLVAQPDVTMEEAENQPAGSVTPVTAGSPGAPELTPLRNKRVSELAGIPGGSARHRGTSSTSSPDSPCTPLSQVGAQQYNLDFLEQFVQRLDTEEGKDCVREIIREMRENQTPELERIRRQACTPVSRKHQRPAPGIPDFCLTPEFQQRMADDFERRWRLPTMKIKPDTPLAVIRQRVMRITCETLGIEYEESNAKTPTLSESPSTVKKKPPTRTTQATKLNFDRSPKTPKLSLGKKTPLRVSMGSPRSGTQSPFVPNTQSPIEAAPPRRSDGPTLSEEGQSTINFDKISFEESAVPVLAPDVPTVAPDVKQITDYLKNCESRRNSLKRSHDNDMDCGESEVQYVQPFESEGFALGTEDMVDWRDPAEFNAAKRRSSGGSPKMQYAGIPCFSISCGDDDEKRAELIARITQLGGKVCENLVNYDDSCTHLLCERPNRGEKMLACIAAGKWILNIQYIEQSHARGDFLDETLYEWGNPKAINLPTLAPEEEPIAAAVHRWRTELSACGGGAFSDHRVILSMNERSGAPIRNVLRAGGACILEPTTPFSKDPVAKSASHCFVDVKKAPLSTQDMEYLHKCGVQVLSQIAINAYLMNGRDADLGKYQLL

SEQ ID NO: 3 - protein, 14 aa, Drosophila sp.
AMRDLQVSATGITP

SEQ ID NO: 4 - protein, 16 aa, Drosophila sp.
KEELSRLINWMGGIYF

SEQ ID NO: 5 - protein, 11 aa, Drosophila sp.
HRTTHLISNTI

SEQ ID NO: 6 - protein, 27 aa, Drosophila sp.
GVPVMHVDWVQYVWDQSRRSQREGIMA

SEQ ID NO: 7 - protein, 14 aa, Homo sapiens
VMSDVTISCTSLEK

SEQ ID NO: 8 - protein, 16 aa, Homo sapiens
REEVHKYVQMMGGRVY

SEQ ID NO: 9 - protein, 11 aa, Homo sapiens
VSVTHLIAGEV

SEQ ID NO: 10 - protein, 25 aa, Homo sapiens
KKPILLPSWIKTLWEKSQEKKITRY

SEQ ID NO: 11 - protein, 14 aa, Caenorhabditis elegans
VFQDVKISFTGLNL

SEQ ID NO: 12 - protein, 16 aa, Caenorhabditis elegans
KQELYEKIGWMCGVVG

SEQ ID NO: 13 - protein, 11 aa, Caenorhabditis elegans
HETTHLVTEKA

SEQ ID NO: 14 - protein, 27 aa, Caenorhabditis elegans
SIKLMRIGWIDDLWETSQTTMGRFSAL

SEQ ID NO: 15 - protein, 14 aa, Schizosaccharomyces pombe
LKGFVICCTSIDLK

SEQ ID NO: 16 - protein, 16 aa, Schizosaccharomyces pombe
RTEISTKATKLGAAYR

SEQ ID NO: 17 - protein, 11 aa, Schizosaccharomyces pombe
KDVTHLIAGDF

SEQ ID NO: 18 - protein, 27 aa, Schizosaccharomyces pombe
WIPVLYESWVQGEDLDDGLLVDKHFLP

SEQ ID NO: 19 - protein, 13 aa, Mus sp.
MLNLVLCFTGFRK

SEQ ID NO: 20 - protein, 16 aa, Mus sp.
LVKLVTLVHHMGGVIR

SEQ ID NO: 21 - protein, 11 aa, Mus sp.
SKVTHLVANCT

SEQ ID NO: 22 - protein, 27 aa, Mus sp.
GTPIMKPEWIYKAWERRNEQCFCAAVD

SEQ ID NO: 23 - protein, 723 aa, Drosophila sp.
  SITE (212)..(227): Xaa is uncertain
ILGPPCLITCLRRNEPIPEGSSAIYSTAMRDLQVSATGITPQKKEELSRLINWMGGIYFQSFGHRTTHLISNTIKSSKYEQATLNGVPVMHVDWVQYVWDQSRRSQREGIMATDPDFDKYRLPIFFGANITCSGLDVARKDQVMRLVNDNGGIYHRAFRSQVVDIVITEQTKTDTEKYKAAIRYKKDVLLPEWIFDSCNRGYALPTKDYEVXXXXXXXXXXXXXXXXAAPGADQTHLSDLSRISFVSGSRRMCSDLXXXXXXXXXXXXXXPAKQLLKQATSSGRNYQQVLAEIEPRQAKKAGAFLDGCCVYLSGFRSEEREKLNRVLNTGGATRYDEANEGISHIIVGQLDDAEYRQWQRDGLMGSVHVVRLDWLLESIRAGRVVSELVHRVSMPQNREPDVASPASKRTLRSMNHSFKQPTLPIKKKLFDQEPDPVQEQEHEEPDHTLLDQYSQDQGAVAQLPPADVSLLQPAASSTQMDIRQRVSVANPKPPAEGLQLPDLSASTLSIDFDKLDYFAGVSVYVHRECFNEEFFNQMLTECEAAQGLLVPSSFSDEVDFAIVSFEVAFDVKQLPVKARHVVTELFLESCMKKNQLLPIEYYHKHVPATALRQPLKGMTIVVSIYAGLERDFINATAELLGASVNKTFIKKEKPLLVCPSAEGSKYEGAIKWNYPVVTSDWLVQCARTGQKLPFVGYLVGKSPEDFPISPRLRDSNSRTARRP

SEQ ID NO: 24 - protein, 689 aa, Homo sapiens
IVGPQVVIFCMHHQRCVPRAEHPVYNMVMSDVTISCTSLEKEKREEVHKYVQMMGGRVYRDLNVSVTHLIAGEVGSKKYLVAANLKKPILLPSWIKTLWEKSQEKKITRYTDINMEDFKCPIFLGCIICVTGLCGLDRKEVQQLTVKHGGQYMGQLKMNECTHLIVQEPKGQKYECAKRWNVHCVTTQWFFDSIEKGFCQDESIYKTEPRPEAKTMPNSSTPTSQINTIDSRTLSDVSNISNINASCVSESICNSLNSKLEPTLENLENLDVSAFQAPEDLLDGCRIYLCGFSGRKLDKLRRLINSGGGVRFNQLNEDVTHVIVGDYDDELKQFWNKSAHRPHVVGAKWLLECFSKGYMLSEEPYIHANYQPVEIPVSHQPESKAALLKKKNSSFSKKDFAPSEKHEQADEDLLSQYENGSSTVVEAKTSEARPFNDSTHAEPLNDSTHISLQEENQSSVSHCVPDVSTITEEGLFSQKSFLVLGFSNENESNIANIIKENAGKIMSLLSRTVADYAVVPLLGCEVEATVGEVVTNTWLVTCIDYQTLFDPKSNPLFTPVPVMTGMTPLEDCVISFSQCAGAEKESLTFLANLLGASVQEYFVRKSNAKKGMFASTHLILKERGGSKYEAAKKWNLPAVTIAWLLETARTGKRADESHFLIENSTKEERSLETEITNGINLNSDTAEHP

SEQ ID NO: 25 - protein, 194 aa, Drosophila sp.
EKRAELIARITQLGGKVCENLVNYDDSCTHLLCERPNRGEKMLACIAAGKWILNIQYIEQSHARGDFLDETLYEWGNPKAINLPTLAPEEEPIAAAVHRWRTELSACGGGAFSDHRVILSMNERSGAPIRNVLRAGGACILEPTTPFSKDPVAKSASHCFVDVKKAPLSTQDMEYLHKCGVQVLSQIAINAYLM

SEQ ID NO: 26 - protein, 187 aa, Homo sapiens
QERIDYCHLIEKLGGLVIEKQCFDPTCTHIVVGHPLRNEKYLASVAAGKWVLHRSYLEACRTAGHFVQEEDYEWGSSSILDVLTGINVQQRRLALAAMRWRKKIQQRQESGIVEGAFSGWKVILHVDQSREAGFKRLLQSGGAKVLPGHSVPLFKEATHLFSDLNKLKPDDSGVNIAEAAAQNVYCL

SEQ ID NO: 27 - DNA, 18 bp, Artificial Sequence
  Description of Artificial Sequence Primer
ccgaagctatcgctaggt

SEQ ID NO: 28 - DNA, 18 bp, Artificial Sequence
  Description of Artificial Sequence Primer
agtcccacgcgcatgcga

SEQ ID NO: 29 - DNA, 18 bp, Artificial Sequence
  Description of Artificial Sequence Primer
gacgatagctgcacccat

SEQ ID NO: 30 - DNA, 18 bp, Artificial Sequence
  Description of Artificial Sequence Primer
gtggcggcgctgcttcga

SEQ ID NO: 31 - DNA, 18 bp, Artificial Sequence
  Description of Artificial Sequence Primer
gaaggcgccgctgtcgac

SEQ ID NO: 32 - DNA, 18 bp, Artificial Sequence
  Description of Artificial Sequence Primer
cgcacctctcggatctct

SEQ ID NO: 33 - DNA, 18 bp, Artificial Sequence
  Description of Artificial Sequence Primer
ccacagcttcaagcagcc

SEQ ID NO: 34 - DNA, 18 bp, Artificial Sequence
  Description of Artificial Sequence Primer
aggcaggtgatgaggcac

SEQ ID NO: 35 - DNA, 18 bp, Artificial Sequence
  Description of Artificial Sequence Primer
gtcttcttgctgtccggc

SEQ ID NO: 36 - DNA, 18 bp, Artificial Sequence
  Description of Artificial Sequence Primer
accgttggcaacgctggc

SEQ ID NO: 37 - DNA, 18 bp, Artificial Sequence
  Description of Artificial Sequence Primer
cgccatgtggtcaccgaa

SEQ ID NO: 38 - DNA, 18 bp, Artificial Sequence
  Description of Artificial Sequence Primer
tgtgtggtggtgaccaac

SEQ ID NO: 39 - DNA, 18 bp, Artificial Sequence
  Description of Artificial Sequence Primer
ctcttcgctttggtttag

SEQ ID NO: 40 - DNA, 18 bp, Artificial Sequence
  Description of Artificial Sequence Primer
gccagcgttgccaacggt

SEQ ID NO: 41 - DNA, 18 bp, Artificial Sequence
  Description of Artificial Sequence Primer
ttagcttctcacgctcct

SEQ ID NO: 42 - DNA, 18 bp, Artificial Sequence
  Description of Artificial Sequence Primer
acgatgatagatgcctcc

SEQ ID NO: 43 - DNA, 18 bp, Artificial Sequence
  Description of Artificial Sequence Primer
tggcaccaataagccttg

SEQ ID NO: 44 - DNA, 18 bp, Artificial Sequence
  Description of Artificial Sequence Primer
gagctcctgaactgacgc

SEQ ID NO: 45 - DNA, 18 bp, Artificial Sequence
  Description of Artificial Sequence Primer
tcgcgattcgcagttctt

SEQ ID NO: 46 - DNA, 18 bp, Artificial Sequence
  Description of Artificial Sequence Primer
gtcgacagcggcgccttc

SEQ ID NO: 47 - DNA, 18 bp, Artificial Sequence
  Description of Artificial Sequence Primer
tagcgtaatgaattacta

SEQ ID NO: 48 - DNA, 13 bp, Artificial Sequence
  Description of Artificial Sequence Consensus sequence
cacaaccaaaatg